(19) United States
(12) Patent Application Publication
(10) Pub. No.: US 2026/0202404 A1
(43) Pub. Date: Jul. 16, 2026

(54) Peptides, Reagents And Methods For Detecting Food Allergy

(71) Applicants: AllerGenis, Inc., Wynnewood, PA (US); ICAHN School of Medicine at Mount Sinai, New York, NY (US)

(72) Inventors: Robert C. Getts, Wynnewood, PA (US); James Kadushin, Wynnewood, PA (US); Hugh A. Sampson, Greenwich, CT (US); Ludmilla Bardina, Red Hook, NY (US); Galina Grishina, Tarrytown, NY (US); Gustavo Gimenez, New York, NY (US); Jing Lin, Guangzhou (CN)

(21) Appl. No.: 19/443,288

(22) Filed: Jan. 8, 2026

Related U.S. Application Data
(63) Continuation of application No. 17/536,667, filed on Nov. 29, 2021, now Pat. No. 12,546,775.
(63) Continuation of application No. 15/129,979, filed on Mar. 20, 2015, now Pat. No. 11,215,615.
(60) Provisional application No. 61/974,675, filed on Apr. 3, 2014.

(51) Int. Cl. G01N 33/564; A61K 39/35; C07K 7/00; C07K 7/08; G01N 33/68

(52) U.S. Cl. CPC G01N 33/564; A61K 39/35; C07K 7/00; C07K 7/08; G01N 33/6854; G01N 2800/24

(57) ABSTRACT

Provided are peptide biomarkers for diagnosis of allergy, monitoring development of clinical tolerance in an allergic individual, and predicting whether an allergic subject is likely to develop clinical or natural tolerance over time. The invention also relates to diagnostic methods and diagnostic kits employing the peptide biomarkers.

### CROSS REFERENCE TO SEQUENCE LISTING

This application includes a Sequence Listing submitted electronically as an XML file named 896983060SEQ, created on Jan. 6, 2026, with a size of 597,699 bytes. The Sequence Listing is incorporated herein by reference.

### TECHNICAL FIELD

The invention relates to peptide biomarkers for diagnosis of allergy and for determining whether an allergic subject is likely to outgrow the allergy. The invention also relates to diagnostic methods and diagnostic kits employing the peptide biomarkers.

### BACKGROUND

Food allergies are a common problem among adults and children, and symptoms may range from mild oral pruritus to potentially life-threatening anaphylactic shock. Food allergies are currently diagnosed by skin prick testing or oral provocation, and measurement of serum levels of specific IgE and in some cases other serum antibodies, such as IgG4. These tests indicate the likelihood of clinical reactivity but do not distinguish the different phenotypes of food allergy or provide prognostic information. They also involve some level of risk to the patient. The relationship between current IgE testing and the actual clinical sensitivity of the patient is a weak one that is usually defined as a combination of reaction severity and the amount of allergen that provokes a reaction. Another limitation of current testing is the inability to determine whether or not pediatric patients will outgrow the allergy during childhood. In this case there is a positive but weak correlation between specific IgE level and the duration of clinical allergy.

More recently, it has been suggested that clinical reactivity to food allergens may correlate better with allergen-specific IgE on the epitope recognition level. It has been reported that patients with persistent or more severe allergic reactions recognize larger numbers of IgE epitopes, suggesting epitope mapping as an additional tool for allergy diagnosis and prediction. Spot membrane-based immunoassays have been used for epitope mapping. In this system, peptides are synthesized on the membrane and incubated with the patient's sera. The process requires a large number of peptides and is therefore error prone, time consuming, labor intensive, and expensive. Immunoassays in this format also require a large volume of patient serum.

Development of multiplex assay technologies, such as microarrays, and advances in peptide synthesis techniques have improved epitope mapping of food allergens. Immunoassays in microarray format can assay thousands of target peptides in parallel using small volumes of diluted serum, greatly reducing the cost and allowing for better replication and statistical approaches to the analysis. Unfortunately, the microarray-based test is not high through-put and it frequently requires multiple replicates to overcome limitations in reproducibility.

High-throughput assay formats have the advantage of rapidly processing multiple patient specimens in an automated fashion, and have been developed for application to multiplex screening methods. Bead-based multiplexing, such as the LUMINEX/xMAP technology, uses 5.6 μm polystyrene beads dyed with red and infrared fluorophores. Using different amounts of each of the two fluorophores, up to 500 different specific spectral signatures can be produced and theoretically up to 500 tests in a single reaction volume is possible. In the typical protein assay, antibodies are conjugated to the surface of the beads to capture the analyte of interest. Biotinylated detection antibodies specific to the analyte of interest are then bound to form an antibody-antigen sandwich. Interaction of biotin with a phycoerythrin-conjugated streptavidin (SA-PE) is used to label the complex. To detect the analyte, the beads are read on a dual-laser flow-based detection instrument. One laser classifies the bead according to the incorporated dyes and determines the analyte that is being detected. The second laser determines the magnitude of the PE-derived signal, which is in direct proportion to the amount of the bound analyte. Such assays reduce the amount of capture antibody and sample required compared to an ELISA plate assay, thus reducing the cost and conserving rare or difficult-to-obtain sample material. The dynamic range and sensitivity of the assay are also generally improved.

Cow's milk allergy (CMA) is one of the most common food allergies in children. It typically involves sensitivity to several of the component proteins of cow's milk. These include proteins in the casein fraction (αₛ₋₁-, αₛ₋₂-, ß-, and κ-casein), α-lactalbumin and ß-lactoglobulin. Both conformational and sequential epitopes can elicit antibody responses. Although the majority of children eventually outgrow their CMA (i.e., they become clinically tolerant), some retain their sensitivity into later life. The mechanisms contributing to development of clinical tolerance are not well understood, but it is hypothesized that IgE antibodies of those with persistent CMA may recognize certain epitopes of cow's milk proteins that are not recognized by IgE antibodies from patients who are likely to outgrow their allergy.

Analysis of epitopes, such as sequential epitope recognition, can provide useful information concerning persistence of CMA. Peptide microarray results have shown a correlation with clinical features of milk allergy, i.e., patients with milk allergy and milk-tolerant patients evidenced different epitope recognition patterns. It was also demonstrated that changes in the relative binding of IgE and IgG4 to milk peptides correlated with the presence of allergy or with clinical improvement.

However, there remains a need to identify informative epitopes that are useful for diagnosing CMA and for predicting the clinical outcome of CMA. There also remains a need for new assay platforms that overcome the deficiencies of microarray immunoassays, and provide high-throughput, increased flexibility, reduced sample volume, and lower cost, with a similar workflow. The present invention addresses these needs.

### SUMMARY

In a first embodiment, the invention relates to peptides containing allergenic epitopes of cow's milk proteins that are useful for diagnosis of CMA, for detecting development of clinical tolerance to cow's milk proteins, and for monitoring increases and decreases in the intensity of the allergic response.

In a specific aspect of the first embodiment, the allergenic epitope-containing peptides are a plurality of peptides selected from the group consisting of allergenic peptide epitopes of αS1-casein, αS2-casein, ß-casein, ß-lactoglobulin and κ-casein. In a further specific embodiment, the allergenic epitope-containing peptides are a plurality of peptides selected from among SEQ ID NOs: 1-33:

AlphaS-1 Casein Peptides:

| | | |
|---|---|---|
| a1phaS1-03 | KHQGLPQEVLNENLLRFFVA | SEQ ID NO: 1 |
| alphaS1-09 | VAPFPEVFGKEKVNELSKDI | SEQ ID NO: 2 |
| alphaS1-22 | SISSSEEIVPNSVEQKHIQK | SEQ ID NO: 3 |
| alphaS1-27 | KHIQKEDVPSERYLGYLEQL | SEQ ID NO: 4 |
| alphaS1-30 | SERYLGYLEQLLRLKKYKVP | SEQ ID NO: 5 |
| alphaS1-35 | KYKVPQLEIVPNSAEERLHS | SEQ ID NO: 6 |
| alphaS1-44 | QQKEPMIGVNQELAYFYPEL | SEQ ID NO: 7 |
| alphaS1-57 | LGTQYTDAPSFSDIPNPIGS | SEQ ID NO: 8 |
| alphaS1-61 | SDIPNPIGSENSEKTTMPLW | SEQ ID NO: 9 |

AlphaS-2 Casein Peptides:

| | | |
|---|---|---|
| alphaS2-08 | QEKNMAINPSKENLCSTFCK | SEQ ID NO: 10 |
| alphaS2-13 | STFCKEVVRNANEEEYSIGS | SEQ ID NO: 11 |
| a1phaS2-26 | KHYQKALNEINQFYQKFPQY | SEQ ID NO: 12 |
| a1phaS2-33 | QYLYQGPIVLNPWDQVKRNA | SEQ ID NO: 13 |
| a1phaS2-56 | KISQRYQKFALPQYLKTVYQ | SEQ ID NO: 14 |
| a1phaS2-60 | QYLKTVYQHQKAMKPWIQPK | SEQ ID NO: 15 |

Beta-Casein Peptides:

| | | |
|---|---|---|
| betacas-01 | RELEELNVPGEIVESLSSSE | SEQ ID NO: 16 |
| betacas-16 | QDKIHPFAQTQSLVYPFPGP | SEQ ID NO: 17 |
| betacas-18 | FAQTQSLVYPFPGPIPNSLP | SEQ ID NO: 18 |
| betacas-25 | NIPPLTQTPVVVPPFLQPEV | SEQ ID NO: 19 |
| betacas-33 | KVKEAMAPKHKEMPFPKYPV | SEQ ID NO: 20 |
| betacas-42 | SLTLTDVENLHLPLPLLQSW | SEQ ID NO: 21 |
| betacas-53 | FPPQSVLSLSQSKVLPVPQK | SEQ ID NO: 22 |
| betacas-58 | PVPQKAVPYPQRDMPIQAFL | SEQ ID NO: 23 |

Beta-Lacto globulin Peptides:

| | | |
|---|---|---|
| betalac-14 | RVYVEELKPTPEGDLEILLQ | SEQ ID NO: 24 |
| betalac-22 | DECAQKKIIAEKTKIPAVFK | SEQ ID NO: 25 |
| betalac-41 | CLVRTPEVDDEALEKFDKAL | SEQ ID NO: 26 |
| betalac-43 | EVDDEALEKFDKALKALPMH | SEQ ID NO: 27 |

Kappa Casein Peptides:

| | | |
|---|---|---|
| kappacas-04 | RCEKDERFFSDKIAKYIPIQ | SEQ ID NO: 28 |
| kappacas-16 | KPVALINNQFLPYPYYAKPA | SEQ ID NO: 29 |
| kappacas-36 | MAIPPKKNQDKTEIPTINTI | SEQ ID NO: 30 |
| kappacas-44 | PTSTPTTEAVESTVATLEDS | SEQ ID NO: 31 |
| kappacas-49 | TLEDSPEVIESPPEINTVQV | SEQ ID NO: 32 |
| kappacas-21 | YAKPAAVRSPAQILQWQVLS | SEQ ID NO: 33 |

Peptides useful in methods for diagnosis of CMA, for detecting development of clinical tolerance to cow's milk proteins, and for detecting increases and decreases in the intensity of the allergy may also include peptides containing non-reactive epitopes of cow's milk proteins. These peptides are useful as negative controls. In specific aspects the peptides containing negative control epitopes are one or more peptides selected from the group consisting of non-reactive peptide epitopes of αS2-casein, ß-casein, and ß-lactoglobulin. In a further specific embodiment, the non-reactive epitope-containing peptides are one or more peptides selected from the group consisting of:

AlphaS-2 Peptides:

| | | |
|---|---|---|
| a1phas2-42 | NREQLSTSEENSKKTVDMES | SEQ ID NO: 34 |

Beta-Casein Peptides:

| | | |
|---|---|---|
| betacas-09 | RINKKIEKFQSEEQQQTEDE | SEQ ID NO: 35 |

Beta-Lactoglobulin Peptides:

| | | |
|---|---|---|
| betalac-31 | KVLVLDTDYKKYLLVCMENS | SEQ ID NO: 36 |

In a second embodiment, the invention relates to methods for diagnosing CMA using a plurality (i.e., two or more) of the foregoing allergenic epitope-containing peptides. In specific aspects, CMA in a subject is diagnosed by a method comprising:
a) providing a plurality of peptides selected from among SEQ ID NOs: 1-33, each peptide conjugated to a separately identifiable solid support;
b) contacting each solid support with serum obtained from the subject under conditions sufficient to permit binding of allergy-associated immunoglobulin (AAI) in the serum to the peptide on each solid support to form a peptide-AAI complex;
c) binding an AAI-specific labeling reagent to the peptide-AAI complex; and
d) analyzing binding of the labeling reagent to each peptide-AAI complex to identify peptides recognized by the AAI in the serum of the subject;
   wherein recognition of at least one peptide by the AAI in the serum of the subject indicates that the subject is allergic to cow's milk.

In a further embodiment, the invention relates to methods for detecting development of clinical tolerance in a subject having CMA using a plurality of the foregoing allergenic epitope-containing peptides. In specific aspects, development of clinical tolerance to cow's milk in a subject having CMA is detected by a method comprising:
a) providing an initial profile of allergy associated immunoglobulin (AAI) reactivity in the subject's serum to a plurality of peptides selected from among SEQ ID NOs: 1-33, wherein the initial profile defines an initial number of peptides recognized by AAI in the serum of the subject or an initial concentration of AAI in the serum of the subject that recognizes each peptide;
b) providing the plurality of peptides selected from among SEQ ID NOs: 1-33, each peptide conjugated to a separately identifiable solid support
b) contacting each solid support with serum obtained from the subject at a time-point subsequent to the initial profile under conditions sufficient to permit binding of AAI in the serum to the peptide on each solid support to form a peptide-AAI complex;
c) binding an AAI-specific labeling reagent to the peptide-AAI complex; and
d) analyzing binding of the labeling reagent to each peptide-AAI complex to identify a subsequent number of peptides recognized by AAI in the serum of the subject or a subsequent concentration of AAI in the serum of the subject that recognizes each peptide;
wherein development of clinical tolerance to cow's milk is indicated when the subsequent number of peptides recognized by AAI in the serum of the subject is less than the initial number of peptides recognized by AAI in the serum of the subject, or when the subsequent concentration of AAI in the serum of the subject that recognizes at least one peptide is less than the initial concentration of AAI in the serum of the subject that recognizes the at least one peptide.

In another embodiment, the initial detection of development of clinical tolerance is used to predict if a patient will either develop a natural tolerance to the allergy or be responsive to therapy. In this embodiment, an allergic subject is exposed to the immunogen (immunotherapy) prior to analyzing the initial profile. If at the subsequent time-point there is a reduction of at least 2-fold in serum concentration of all AAIs that were highly reactive with peptides in the initial profile, it is likely that the subject will develop either clinical or natural tolerance to cow's milk. If at the subsequent time-point there is a reduction of at least 2-fold in serum concentration of fewer than all AAIs that were highly reactive with peptides in the initial profile, the subject is likely to develop only partial clinical or natural tolerance to cow's milk.

In an alternative embodiment, the methods of the invention can be used to detect an increase (or decrease) in the intensity of the allergic response to cow's milk (CMA intensity) in a subject over a period of time. In specific aspects, detection of an increase in intensity of the allergic response may correspond to development of CMA in a previously cow's milk-tolerant subject. Alternatively, detection of an increase in intensity of the allergic response may correspond to an increase in allergy intensity in a subject previously known to have CMA. Detection of a decrease in the intensity of the allergic response to cow's milk is an aspect of development of clinical tolerance to cow's milk proteins, as discussed above.

In specific aspects, an increase in intensity of allergy to cow's milk in a subject over time is detected by a method comprising:
a) providing an initial profile of allergy associated immunoglobulin (AAI) reactivity in the subject's serum to a plurality of peptides selected from among SEQ ID NOs: 1-33, wherein the initial profile defines an initial number of peptides recognized by AAI in the serum of the subject or an initial concentration of AAI in the serum of the subject that recognizes each peptide;
b) providing the plurality of peptides selected from among SEQ ID NOs: 1-33, each peptide conjugated to a separately identifiable solid support
b) contacting each solid support with serum obtained from the subject at a time-point subsequent to the initial profile under conditions sufficient to permit binding of AAI in the serum to the peptide on each solid support to form a peptide-AAI complex;
c) binding an AAI-specific labeling reagent to the peptide-AAI complex; and
d) analyzing the binding of the labeling reagent to each peptide-AAI complex to identify a subsequent number of peptides recognized by AAI in the serum of the subject or a subsequent concentration of AAI in the serum of the subject that recognizes each peptide;
wherein an increase in the subsequent number of peptides recognized by AAI in the serum of the subject compared to the initial number of peptides recognized by AAI in the serum of the subject, or an increase in the subsequent concentration of AAI in the serum of the subject that recognizes at least one peptide compared to the initial concentration of AAI in the serum of the subject that recognizes the at least one peptide, indicates increased intensity of the allergic response to cow's milk in the subject.

Any of the foregoing embodiments and aspects of the methods of the invention may be in the form of a microarray immunoassay, wherein each of the plurality of allergenic epitope-containing peptides is bound to a separate well of a microtiter plate and reacted with serum to bind AAI. Bound AAI is detected by binding of an AAI specific labeling reagent, for example an anti-AAI antibody conjugated to a reporter moiety such as a fluorescent label. Fluorescence of the bound labeling reagent indicates presence of in the serum of antibody to the allergenic epitope contained in the peptide bound to the well. The plurality of allergenic epitope-containing peptides may also be used in a lateral flow immunoassay format, wherein each peptide is immobilized in a discrete area on a porous or chromatographic support, and the serum is wicked through the support to contact the peptides for binding of AAI to the peptides. In this assay, the AAI specific labeling reagent may comprise a chromophore or dye conjugated to anti-AAI antibody. The labeling reagent is also wicked through the support to contact the peptide-AAI complexes for binding of the labeling reagent to the complex, which indicates the presence or absence in the serum of antibody to the allergenic epitope contained in the peptide immobilized at each discrete location of the support.

In an alternative aspect, any of the foregoing embodiments and aspects of the methods of the invention may be in the form of a flow cytometry assay in which each allergenic epitope-containing peptide is conjugated to a separately identifiable solid support suitable for analysis by flow cytometry, such as a bead. Typically, the peptide is conjugated to the solid support by binding to a peptide-specific capture antibody on the solid support or by chemical linkage to the solid support. In this aspect, the bead with the conjugated allergenic epitope-containing peptide is contacted with the serum of a subject to bind any peptide-specific AAI that is present to the bead, forming a peptide-AAI complex on the bead. An AAI-specific labeling reagent comprising a fluorescent reporter moiety is then bound to the peptide-AAI complexes and the beads are analyzed quantitatively or qualitatively by flow cytometry. This detects fluorescence from the bound labeling reagent associated with each bead to which an allergenic epitope-containing peptide is conjugated, thereby identifying the peptide and the presence in the serum of AAI that is reactive to it. Presence of AAI reactive to at least one of a plurality of allergenic epitope-containing peptides selected from among SEQ ID NOs: 1-33 indicates that the subject is allergic to cow's milk, and changes over time in the number of reactive peptides, or changes over time in the concentration of AAI reactive to one or more peptides, indicates an increase in intensity of the allergy, a decrease in the intensity of the allergy, or development of clinical tolerance over that time period.

In a further aspect, the flow cytometry assay may be a multiplex assay, such at the LUMINEX xMAP technology, which uses a microsphere array platform for quantitation and detection of peptides and proteins. Each of the plurality of allergenic epitope-containing peptides is bound to a set of beads with different spectral properties which can be used to identify the associated allergenic epitope-containing peptide by flow cytometry. The sets of beads are then contacted with serum of a subject to bind peptide-recognizing AAI to each bead to form a peptide-AAI complex on the bead, and an AAI-specific labeling reagent comprising a fluorescent reporter moiety is bound to the AAI of the complex. The beads are analyzed by monitoring the spectral properties of each bead and the amount of associated fluorescence from the bound labeling reagent. This process allows identification of the peptide on the bead, and the presence or absence of serum AAI that is reactive to it. Results of the assay are interpreted as discussed above.

In a further embodiment, the invention relates to a kit for detection of CMA, detection of an increase or decrease in CMA intensity, or detection of development of clinical tolerance to cow's milk proteins comprising, packaged together and including instructions for use:
a) a plurality of allergenic epitope-containing peptides selected from among SEQ ID NOs: 1-33;
b) a labeling reagent comprising an anti-allergy associated immunoglobulin (AAI) antibody conjugated to a first reporter moiety; and
c) optionally, a second reporter moiety that specifically binds to the labeling reagent.

The labeling reagent may be conjugated to a first reporter moiety that is directly detectable, such as a fluorescent dye, radiolabel, or colored dye. In specific examples, a phycoerythrin (PE) molecule can be directly coupled to an anti-allergy associated immunoglobulin and used for detection. Alternatively, the first reporter moiety may be a reporter moiety that is indirectly detectable (e.g., an enzyme label of chromogenic dye) and the kit may optionally include a specific binding partner for the first reporter moiety conjugated to a directly detectable label (the second reporter moiety). For example, the kit may include a biotin-conjugated anti-AAI antibody and a streptavidin-conjugated fluorescent dye for detection of the biotin-conjugated anti-AAI.

### BRIEF DESCRIPTION OF THE DRAWINGS

FIG. 1 illustrates serum reactivity over time to the peptide panel of SEQ ID NOs: 1-33 of a cow's milk tolerant individual receiving immunotherapy for CMA.

FIG. 2 illustrates serum reactivity over time to the peptide panel of SEQ ID NOs: 1-33 of an individual with CMA who became desensitized in response to immunotherapy for CMA.

FIG. 3 illustrates serum reactivity over time to the peptide panel of SEQ ID NOs: 1-33 of an individual with CMA who partially responded to immunotherapy for CMA.

In the drawings, peptides identified beginning with “a” represent “alpha”, peptides identified beginning with “b” represent “beta” and peptides identified as beginning with “k” represent “kappa.”

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

As used herein, the terms “allergy associated immunoglobulin” and “AAI” refer to immunoglobulins in sera that mediate hypersensitivity to food allergens. These include one or more of IgE, IgA, and IgG (including IgG4).

As used herein, the terms “reactive”, “reactivity”, “recognize” and the like refer to the ability of an allergy associated immunoglobulin to bind to an allergenic epitope containing peptide. The level of reactivity indicates the concentration of AAI in the serum, with high reactivity associated with higher AAI concentrations and lower reactivity associated with lower AAI concentrations. The relative AAI concentration (i.e., the relative serum reactivity) is determined by the amount of signal detected in the assay. The level of reactivity of AAI to allergenic epitope containing peptides also indicates the intensity of the allergic response, i.e., higher reactivity is associated with a more intense allergic reaction.

As used herein, the term “clinical tolerance” refers to immunological tolerance to a food allergen that is developed by an allergic subject as a result of exposure to the allergen, i.e., tolerance developed as a result of immunotherapy.

As used herein, the term “natural tolerance” refers to immunological tolerance to a food allergen that is developed by an allergic subject as a biochemical process over time, either as a result of natural exposure to the allergen during a lifetime or in the absence of exposure.

It is to be understood that although the allergenic epitope-containing peptides disclosed herein are described as specific embodiments having specific amino acid sequence, one skilled in the art will recognize that each such peptide may be shifted in either the N-terminal or C-terminal direction of the protein from which it is derived to obtain a related peptide sequence that still contains the relevant epitope but in which the relevant epitope is flanked by different amino acids than specified. Accordingly, in all embodiments and aspects the invention includes allergenic epitope containing peptides having amino acid sequences that overlap with the disclosed peptide sequences by 8 or more contiguous amino acids.

The allergenic epitope-containing peptides represented by SEQ ID NOs: 1-33 were identified in a library of peptides derived from αS1-casein, αS2-casein, ß-casein, ß-lactoglobulin, and κ-casein as having a z-score in highly allergic individuals of greater than 10. In highly allergic subjects, all thirty-three peptides of SEQ ID NOs: 1-33 are reactive with sera. Conversely, in non-allergic subjects none of the thirty-three peptides of SEQ ID NOs: 1-33 are reactive with sera. The individual peptides of SEQ ID NOs: 1-33 also provide a continuum of reactivity which is useful for determining the intensity of CMA in an individual, and for monitoring changes in the intensity of CMA over time. Individuals having intensities of allergy to cow's milk that fall between non-reactive and the most highly reactive have sera that are reactive with some, but not all, of the peptides among SEQ ID NOs: 1-33. In general, the number of peptides among SEQ ID NOs: 1-33 that are reactive with the sera of these individuals is positively correlated with the intensity of the allergy, i.e., the more intense the allergy the more peptides among SEQ ID NOs: 1-33 are reactive with the sera. The sera of individuals with mild allergy are reactive with fewer peptides than the sera of individuals with more intense allergy. The invention therefore not only provides methods for diagnosing CMA, it provides methods for determining the intensity of the allergy and methods for determining changes in the intensity of the allergy over time, including detection of development of clinical tolerance to cow's milk proteins.

In certain aspects of the invention, the number of allergenic epitope-containing peptides within the group of SEQ ID NOs: 1-33 that are reactive with the sera of a CMA subject has a positive correlation with the intensity of the allergic response, i.e., reactivity with fewer peptides indicates a milder allergic response to cow's milk and reactivity with more peptides indicates the subject is more highly allergic to cow's milk. In another aspect of the invention, the intensity of binding of serum IgE to the peptides represented by SEQ ID NOs: 1-33 (a measure of IgE concentration in the sera) correlates with the intensity of the allergic response, i.e., weaker reactivity with all thirty-three peptides, or with a subset of the thirty-three peptides, indicates a more moderate allergic response compared to stronger reactivity with all thirty-three peptides or with the subset of peptides. As used herein, reference to “non-reactive” or “negative” reactivity with an allergenic epitope-containing peptide means a signal-to-noise ratio (S/N) in the assay that is less than about 2. A typical background signal (N) is that generated by a pool of sera from non-allergenic individuals. Alternatively, the invention contemplates use of negative peptides as the basis for establishing the background signal. As used herein, reference to “weak” or “moderate” “moderate” reactivity with an allergenic epitope-containing peptide means a S/N of about 2-10, although this value may vary depending on the peptide and the allergy. As used herein, reference to “high” or “strong” reactivity with an allergenic epitope-containing peptide means a S/N of greater than about 10.

Previously known assays for CMA based on analysis of peptide epitopes in cow's milk proteins are competitive immunoassays which rely on analysis of the relative affinity of binding of IgE and IgG4 to the epitope. The affinity of antibody binding is believed to be related to whether or not the subject will develop clinical tolerance to cow's milk. In contrast, in one aspect, the present invention is based on an analysis of the presence or absence of AAI binding to each individual peptide in a set of key cow's milk protein epitopes that correlates with a diagnosis of CMA, with the intensity of the allergic response, and with the potential of a patient to either develop tolerance or experience an increased allergic response based on the number of epitopes (i.e., peptides) bound by IgE in the serum of the subject. In a second aspect, the invention is based on analysis of the concentration of AAIs in sera that are reactive with each of the allergenic epitope-containing peptides, which also correlates with the intensity of the allergic response.

One embodiment of the invention relates to a method for diagnosing CMA in a subject comprising providing a plurality of peptides selected from among SEQ ID NOs: 1-33, each peptide conjugated to a separately identifiable solid support, contacting each solid support with serum obtained from the subject under conditions sufficient to permit binding of AAI in the serum to the peptide on each solid support to form a peptide-AAI complex, binding an AAI-specific labeling reagent to the peptide-AAI complex, and analyzing binding of the labeling reagent to each peptide-AAI complex to identify peptides recognized by the AAI in the serum of the subject. If, following exposure to cow's milk allergens, at least one peptide is moderately or highly reactive with serum AAI (S/N>2) and reactivity of one or more of the reactive peptides does not decrease at least 2-fold within about six months, the subject is diagnosed as having CMA.

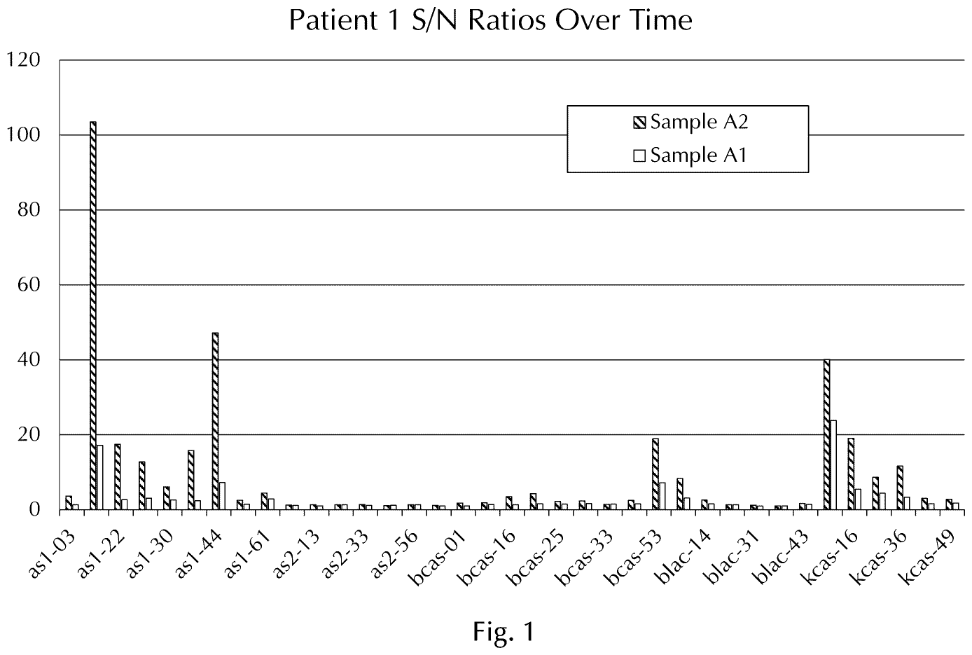

Serum reactivity of a cow's milk tolerant individual following administration of CMA immunotherapy is shown in FIG. 1. In this experiment, a cow's milk tolerant individual was treated with immunotherapy for CMA and serum samples were taken 6-12 months apart. It can be seen that the initial response to immunotherapy resulted in moderate to high reactivity with about eleven of the peptides (blue bars, S/N>2). Within six months (orange bars), there was at least about a 2-fold reduction in reactivity for all of these peptides. Although kcas-04 was in the range of slightly less than a 2-fold reduction in reactivity, the most highly reactive peptides (e.g., as1-09, as1-44) exhibited reductions in reactivity within six months that were substantially larger than 2-fold, in the range of at least 5-fold. In addition, none of the reactive peptides (S/N>2) failed to diminish in reactivity at the six month time-point.

In another aspect of the method, the analysis of binding of the labeling reagent to each peptide-AAI complex may include analysis of the extent of binding, which indicates a concentration of each peptide-specific AAI in the serum. A low to moderate serum reactivity with all of the peptides of SEQ ID NOs: 1-33, or with a subset thereof, indicates a lower concentration of peptide-specific AAI in the serum and mild to moderate CMA, whereas high serum reactivity with all of the peptides, or a subset thereof, indicates a higher concentration of peptide-specific AAI in the serum and more severe CMA. The analysis of binding for diagnosis of CMA may employ either the number of peptides reactive with sera, the extent of binding of serum AAI to the peptides, or both.

In certain aspects, the invention further relates to peptides of cow's milk proteins that contain epitopes that are non-reactive with the sera of subjects that are allergic to cow's milk, even if the subject is phenotypically highly allergic. The sera of non-allergic subjects are also non-reactive with these peptides. These peptides are represented by SEQ ID NOs: 34, 35 and 36, and are useful as negative controls in specific embodiments of the assays for diagnosis of CMA. Having a highly reliable negative control available for this purpose reduces the likelihood of false positive diagnoses and falsely high determinations of reactive AAI concentration.

In specific embodiments, of the methods for diagnosing CMA in a subject using a plurality (two or more) of peptides selected from among SEQ ID NOs: 1-33 include solid phase assays. The plurality of peptides selected for use in the solid phase assay may represent all 33 peptides of SEQ ID NOs: 1-33, a subset of 5-10 peptides, a subset of 10-15 peptides, or a subset of 15-20 peptides. The methods may also employ two or more such subsets of the peptides. Each of the plurality of peptides selected from among SEQ ID NOs: 1-33 is provided conjugated to a solid support, which may be a bead, a microtiter plate, a chromatographic material (e.g., a filter), or any other suitable solid support. Each bead, microtiter plate well, or discrete location on the chromatographic material is occupied by a single peptide selected from among SEQ ID NOs: 1-33. The solid supports are then contacted with serum obtained from the subject under conditions appropriate for specific binding of anti-peptide AAIE in the serum (if present) to the peptide on each solid support or discrete location on a solid support to form a peptide-AAI complex on the solid support.

Any peptide-AAI complex formed on a solid support is then detected by contacting the complex on each solid support or discrete location on the solid support with a labeling reagent that specifically binds to the complex, typically by binding to the immobilized serum AAI antibody. A single labeling reagent will generally be used for universal detection of all complexes. The specific peptide-AAI complex may then be identified by its position on the microtiter plate or chromatographic support. When the solid support to which each peptide is conjugated has different spectral properties, the specific peptide-AAI complex may also be identified by analysis of the spectral properties of the solid support associated with the peptide-AAI complex, once the presence of a complex is identified via a detectable signal from the labeling reagent bound to the complex. As an example, the presence or absence of a peptide-AAI complex in each well of a microtiter plate can be determined by binding to the complex an anti-human AAI antibody that is conjugated to a reporter moiety, such as a fluorescent dye, a chromogenic dye, an enzyme label or a radioactive label. Alternatively, the anti-human AAI antibody may be conjugated to a reporter moiety that is not directly detectable, so specific binding of a second, directly detectable reporter moiety to the labeling reagent is necessary for analysis of binding.

In certain aspects, the methods for diagnosis of CMA are qualitative methods, i.e., based only on presence or absence of AAI reactive to each selected peptide. Presence of AAI moderately or highly reactive with any selected peptide can be considered to indicate some degree of CMA, provided that the reactivity does not substantially diminish within a short period of time such as about six months. The methods may also be semi-quantitative, i.e., the greater the number of peptides reactive with the serum of the subject the relatively more intense the allergy and, conversely, the fewer the number of reactive peptides the relatively less intense the allergy. Serum reactivity with 5-15 of the peptides of SEQ ID NOs: 1-33 may indicate mild to moderate CMA, with reactivity within the lower end of this range generally characterized as mild CMA. Serum reactivity with 16-33, 16-30, 16-25, 16-20, 16-18 or all 33 peptides of SEQ ID NOs: 1-33 may indicate moderate to severe CMA, with reactivity within the lower end of this range generally characterized as moderate CMA. In the midrange, serum reactivity with 10-20, 12-18 or 14-16 of the peptides of SEQ ID NOs: 1-33 may generally be considered to indicate moderate CMA. It is a particularly useful feature of the peptides of SEQ ID NOs: 1-33 that generally no more than about 8-10 are highly reactive (S/N>10) with the sera of non-allergic individuals and thus provide a higher confidence level in the result of the diagnostic assay than conventional assays.

In other aspects, the methods for diagnosis of CMA are quantitative methods, i.e., based on quantitation of the level of AAI reactivity to each selected peptide. In this example, the level of reactivity correlates with the amount of labeling reagent bound to the peptide-AAI complex, with higher levels of signal from the reporter moiety indicating a higher concentration of a particular peptide-specific AAI in the serum. To obtain the amount or concentration of reporter moiety bound to a particular peptide-AAI complex, the quantity of fluorescence from a fluorescent dye, intensity of color from a colored or chromogenic dye or from an enzyme label, or quantity of radioactivity from a radioactive label is positively correlated with the amount of bound AAI in the complex and therefore its concentration. Methods for measuring these parameters are known in the art. The relative quantities of AAI reactive with any of the peptides can be considered to indicate the degree or intensity of CMA. That is, the higher the level of reactivity of the plurality of selected peptides, or of one or more peptides within the selected peptides, the more intense the allergy. Conversely, the lower the level of reactivity of the plurality of selected peptides, or of one or more peptides within the selected peptides, the less intense the allergy.

A particularly useful quantitative assay for use in any of the methods of the invention is a multiplex peptide-bead assay for flow cytometric analysis, such as the LUMINEX exMAP multiplex bead assay, which is a high-throughput alternative to the ELISA. In this assay, polystyrene beads (microspheres) dyed with distinct proportions of red and near-infrared fluorophores are used as the solid support. The peptides may be chemically linked to the beads or bound thereto through peptide-specific capture antibodies coated on the beads. The proportions of the fluorophores define a “spectral address” for each bead population that can be identified by a flow cytometer using digital signal processing. Detection of a third fluorescence color is used for measurement of the fluorescence intensity of the reporter moiety of the labeling reagent bound to the bead. Multiple analytes can be detected simultaneously by binding each peptide selected from among SEQ ID NOs: 1-33 to a bead having a specific “spectral address.” Contacting the beads with serum containing AAI that are specific for the peptide bound to it is followed by addition of anti-human AAI antibodies conjugated to a reporter moiety. In one example, the reporter moiety of the anti-human AAI is biotin and binding to phycoerythyrin (PE)-conjugated streptavidin provides the fluorescent signal for detection. Following binding of the labeling reagent, the beads are analyzed on a dual-laser flow-based detection instrument, such as the LUMINEX 200 or Bio-Rad BIO-PLEX analyzer. One laser classifies the bead and identifies the peptide bound to it. The second laser determines the magnitude of the reporter-derived signal, which is in direct proportion to the amount of bound serum AAI.

Because the degree of binding of each peptide-specific AAI to the peptide-AAI complex on the solid support can be quantitated, the plurality of peptides selected from among peptides represented by SEQ ID NOs: 1-33 are also useful in methods for detecting an increase in the intensity of CMA over time in a subject diagnosed with CMA or development of CMA over time in a subject initially diagnosed as non-allergic. An initial assay is performed on a plurality of peptides selected from among SEQ ID NOs: 1-33 as described above to provide an initial number of reactive peptides or an initial concentration of each peptide-specific AAI. At a time-point subsequent to the initial assay, the analysis is repeated with the same plurality of peptides selected from among SEQ ID NOs: 1-33 as the initial profile to obtain a subsequent number of reactive peptides or a subsequent concentration of peptide-specific AAI. This method can be summarized as follows: providing an initial profile of a subject's serum AAI reactivity to a plurality of peptides selected from among SEQ ID NOs: 1-33, wherein the initial profile indicates an initial number of peptides recognized (bound) by AAI in the serum of the subject or an initial concentration of AAI in the serum of the subject that recognizes (binds to) each peptide; at a time-point subsequent to the initial profile, contacting each peptide of the same plurality of peptides conjugated to a separately identifiable solid support with serum from the subject under conditions sufficient to permit binding of AAI in the serum to the peptide on each solid support, forming a peptide-AAI complex; binding an AAI-specific labeling reagent to the complex, and; analyzing the binding of the labeling reagent to each peptide-AAI complex to identify a subsequent number of peptides recognized by AAI in the serum of the subject or a subsequent concentration of AAI in the serum of the subject that reacts with each selected peptide.

An alternative assay format useful in the invention is a lateral flow or immunochromatographic assay. In such an assay, the selected allergenic epitope containing peptide(s) are immobilized on the porous support and serum containing the AAI is wicked into contact with the peptide(s) to form immunocomplexes. Further migration of the immunocomplex through the porous support brings it into contact with a specific capture reagent for detection of the immunocomplex using appropriate detection reagents.

The methods for detecting an increase in intensity of the allergy may make use of any appropriate assay format, including those described above. Examples of the types of analyses available for analyzing binding of the labeling reagent are also as described above. An increase in the number of peptides reactive with AAI at the subsequent time-point compared to the initial profile (including an increase compared to no peptides reactive with AAI in the initial profile), or an increase in intensity of binding of AAI to any of the peptides at the subsequent time-point compared to the initial profile (including an increase from no binding to a particular peptide in the initial profile to detectable binding at the subsequent time-point), indicates an increase in the intensity of CMA in a subject previously diagnosed with CMA or development of CMA in the previously non-allergic subject. As discussed above, comparing the initial profile of a subject to that of a subsequent time point may be used to predict the subject's increase in severity or lower tolerance in a particular allergy, or to predict the likelihood of development of clinical or natural tolerance to the allergen.

The plurality of peptides selected from among peptides represented by SEQ ID NOs: 1-33 are also useful in methods for detecting development of clinical tolerance to cow's milk proteins in a subject diagnosed with CMA. In these embodiments, the assay generally as described above for detection of an increase in allergy intensity, is performed first at an initial time-point to establish an initial profile of serum AAI reactivity with the plurality of peptides selected from among SEQ ID NOs: 1-33. The initial profile is based on semi-quantitative or quantitative analysis of serum reactivity with the selected peptides, as discussed above. The selected peptides conjugated to the solid supports are then contacted with serum from the subject obtained at a time-point subsequent to the initial profile and the assay is conducted as above with semi-quantitation or quantitation of the intensity of CMA at the subsequent time-point. A reduction in the number of peptides reactive with AAI at the subsequent time-point as compared to the initial profile, or a reduction in intensity of binding of AAI to any of the peptides at the subsequent time-point as compared to the initial profile, particularly at least a 2-fold reduction, indicates development of clinical tolerance to cow's milk proteins. It will be appreciated that development of clinical tolerance to cow's milk proteins in a subject previously diagnosed with CMA also indicates a decrease in allergy intensity over the time period between the initial profile and the subsequent time-point, and that the method can also be used to detect and predict such decreases in allergy intensity over time.

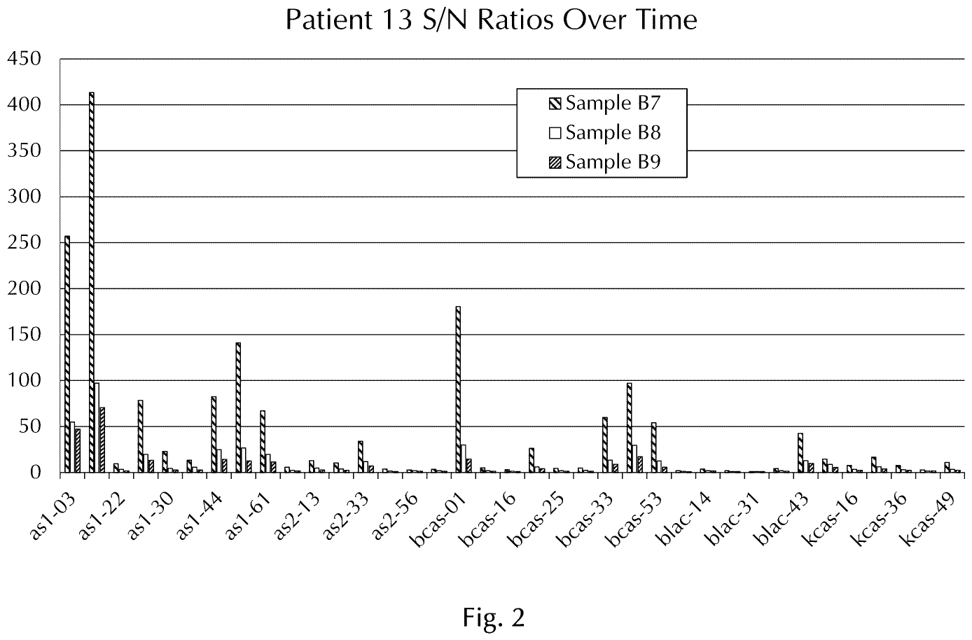

As an example, serum reactivity of a CMA allergic individual is shown in FIG. 2. In this experiment, an individual allergic to cow's milk was treated with immunotherapy for CMA and serum samples were taken 6 months and 12 months later. It can be seen that the initial response to immunotherapy involved moderate to high reactivity with about 15-17 peptides (i.e., S/N>2, blue bars). At six months, serum reactivity had diminished greater than 2-fold for all of the most reactive peptides (orange bars). Reduction in reactivity for certain peptides was in the range of 4-fold to 7-fold (e.g., as1-03, as1-09, as1-57, as1-44, bcas-01). Little or no further reduction in reactivity was observed at 12 months (gray bars), and none of the initially most reactive peptides returned to non-reactive levels (S/N<2). This individual became desensitized to cow's milk over the course of immunotherapy, showing that the assay successfully detected development of clinical tolerance to cow's milk proteins in a subject diagnosed with CMA

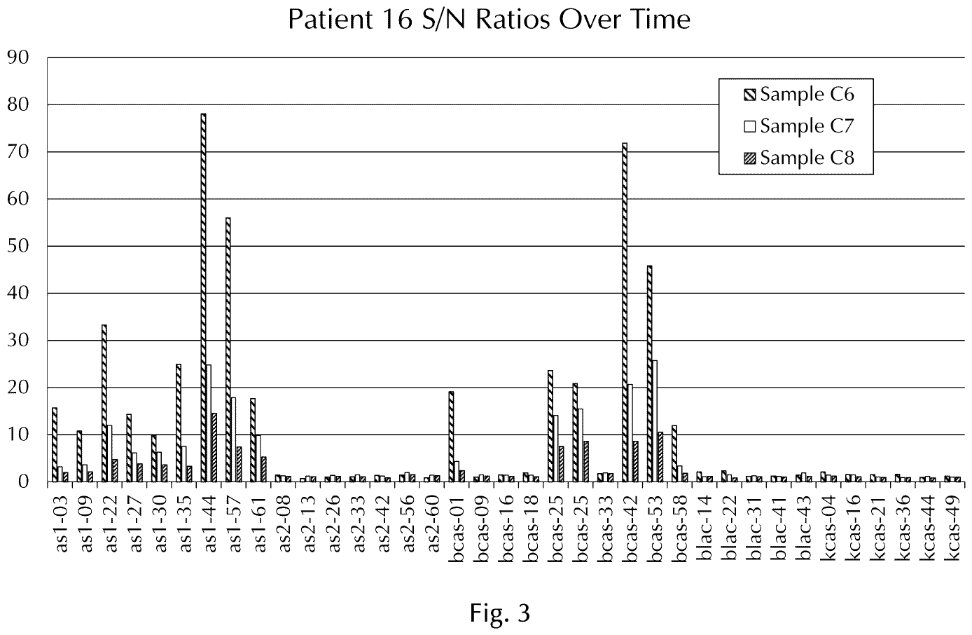

Several peptides in the panel were highly reactive with the sera of the individual shown in FIG. 2 (S/N>10). Similarly, sera of the individual tested in FIG. 3 were moderately to highly reactive with at least about 15 peptides, indicating allergy to cow's milk. In contrast, however, at six months (orange bars) fewer than all of the initially most reactive peptides exhibited a reduction in reactivity of at least 2-fold. Examples of minimal reduction in reactivity <2-fold are seen, for example, with as1-61, bcas-25, and bcas-53. The individual shown in FIG. 3 only partially responded to immunotherapy and, although there were greater than 2-fold reductions in reactivity with some of the most highly reactive peptides, the finding that some of the highly reactive peptides did not exhibit a similar reduction in reactivity may be an indication that immunotherapy is less likely to result in complete desensitization or that complete desensitization may require a longer treatment with immunotherapy.

It will also be recognized that analysis of all thirty-three of the peptides represented by SEQ ID NOs: 1-33 is not always necessary to obtain useful results in the foregoing methods of the invention. It is only necessary to employ a sufficient number of peptides selected from among the peptides represented by SEQ ID NOs: 1-33 to provide a statistically reliable result. For example, if the CMA status of a subject is not known, it is generally desirable to analyze a greater number of allergenic epitope-containing peptides selected from among the peptides represented by SEQ ID NOs: 1-33 to ensure that mild to moderate CMA, that may involve reactivity with only a few of the peptides represented by SEQ ID NOs: 1-33, is detectable. Conversely, if a subject is known to have high-intensity CMA, fewer allergenic epitope-containing peptides selected from among the peptides represented by SEQ ID NOs: 1-33 may be sufficient to detect changes in allergy intensity or development of clinical tolerance, because a larger number of the peptides represented by SEQ ID NOs: 1-33 will be initially reactive. However, because changes in allergy intensity and development of clinical tolerance are evidenced by changes in the number of peptides reactive with sera as well as changes in concentration of serum IgE reactive with a particular peptide, it is particularly desirable to include in the assays a large enough set of peptides selected from among the peptides represented by SEQ ID NOs: 1-33 to ensure that changes with respect to a peptide that is diagnostic for a particular subject are not missed. Accordingly, the plurality of allergenic epitope-containing peptides selected from among peptides represented by SEQ ID NOs: 1-33 for use in any of the foregoing methods may represent all 33 peptides of SEQ ID NOs: 1-33, a subset of 20-25 peptides, a subset of 15-20 peptides, a subset of 10-15 peptides, a subset of 5-10 peptides or a subset of 2-5 peptides. By way of example, it has been found that in many cases the betalac peptides (SEQ ID NOs: 24-27) are substantially less reactive, or non-reactive, with sera of allergic individuals. Accordingly, it may be desirable to use the SEQ ID NOs: 1-23 (the alphaS1, alphaS2, and betacas peptides) alone or with SEQ ID NOs: 28-33 (the kappacas peptides) for certain applications. Each of these subgroups may also be used alone in the invention if desired.

For the convenience of the user, the reagents for use in any of the foregoing methods may be packaged together in the form of a kit comprising a plurality of allergenic epitope-containing peptides selected from among the peptides represented by SEQ ID NOs: 1-33 or any of the useful subgroups, a labeling reagent comprising an anti-human IgE antibody conjugated to a first reporter moiety and, optionally (if required for indirect detection) a second reporter moiety that specifically binds to the labeling reagent. The kit will typically include instructions for use of these reagents in one or more of the methods of the invention described above.

In certain kit embodiments, as well as in the methods of the invention, the anti-human AAI antibody may be provided conjugated to a reporter moiety that can be directly detected. Directly detectable reporter moieties are those that can be identified and/or quantitated without the need for binding to a specific binding partner. Examples of directly-detectable reporter moieties that may be conjugated to the anti-human AAI antibody include fluorescent dyes, colored dyes, chromogenic dyes and enzyme labels that can be detected by a subsequent chemical reaction, and radiolabels. In other kit embodiments, as in the methods of the invention, the anti-human AAI antibody may be provided conjugated to a reporter moiety that is indirectly detectable, i.e., a reporter moiety that is not itself detectable but which undergoes a reaction or interaction with a second reporter moiety that comprises a directly detectable reporter moiety, such as a specific binding partner for the reporter moiety conjugated to a directly detectable label. Examples of indirectly-detectable reporter moieties include biotin, digoxigenin, and other haptens that are detectable upon subsequent binding of a secondary antibody (e.g., anti-digoxigenin) or other binding partner (e.g., streptavidin) which is labeled for direct detection. It will be understood that any of these labeling reagents and reporter moieties are useful in the appropriate assay format in the foregoing methods of the invention and as components of the kits. In a specific example of a kit for performing the flow cytometry multiplex assay described above, the components of the kit may comprise a plurality of allergenic epitope-containing peptides selected from among the peptides represented by SEQ ID NOs: 1-33, a biotinylated anti-human AAI antibody (labeling reagent with first reporter moiety), and streptavidin conjugated to PE (second reporter moiety).

The plurality of allergenic epitope-containing peptides selected from among SEQ ID NOs: 1-33 for inclusion in any of the foregoing kits may represent all 33 peptides of SEQ ID NOs: 1-33, a subset of 20-25 peptides, a subset of 15-20 peptides, a subset of 10-15 peptides, a subset of 5-10 peptides or a subset of 2-5 peptides. The plurality of allergenic epitope-containing peptides selected from among SEQ ID NOs: 1-33 for inclusion in any of the foregoing kits may also represent one or more of the related peptides subgroups (i.e., alphaS1, alphaS2, betacas, betalac and kappacas peptides)

In a further aspect, the invention provides additional allergenic epitope containing peptides derived from cow's milk proteins for use in the foregoing methods, peptide panels and kits. These peptides, and subsets thereof, can be substituted for any or all of SEQ ID NOs: 1-33 in any aspect and/or embodiment discussed above. In addition, the peptides and subsets thereof, can be used in addition to SEQ ID NOs: 1-33 in any aspect and/or embodiment discussed above. The additional allergenic epitope containing peptides derived from cow's milk proteins include:

| | | |
|---|---|---|
| a1phas1-07 | NLLRFFVAPFPEVFGKEKVN | SEQ ID NO: 37 |
| alphas1-25 | PNSVEQKHIQKEDVPSERYL | SEQ ID NO: 38 |
| alphas 1-28 | QKEDVPSERYLGYLEQLLRL | SEQ ID NO: 39 |
| alphas1-37 | LEIVPNSAEERLHSMKEGIH | SEQ ID NO: 40 |
| alphas1-40 | ERLHSMKEGIHAQQKEPMIG | SEQ ID NO: 41 |
| alphas1-43 | IHAQQKEPMIGVNQELAYFY | SEQ ID NO: 42 |
| alphas1-46 | IGVNQELAYFYPELFRQFYQ | SEQ ID NO: 43 |
| alphas1-54 | PSGAWYYVPLGTQYTDAPSF | SEQ ID NO: 44 |
| alphas1-55 | AWYYVPLGTQYTDAPSFSDI | SEQ ID NO: 45 |
| alphas2-05 | SIISQETYKQEKNMAINPSK | SEQ ID NO: 46 |
| alphas2-10 | INPSKENLCSTFCKEVVRNA | SEQ ID NO: 47 |
| alphas2-21 | SAEVATEEVKITVDDKHYQK | SEQ ID NO: 48 |
| alphas2-44 | TSEENSKKTVDMESTEVFTK | SEQ ID NO: 49 |
| alphas2-51 | TKLTEEEKNRLNFLKKISQR | SEQ ID NO: 50 |
| alphas2-62 | YQHQKAMKPWIQPKTKVIPY | SEQ ID NO: 51 |
| betacas-21 | PFPGPIPNSLPQNIPPLTQT | SEQ ID NO: 52 |
| betacas-27 | QTPVVVPPFLQPEVMGVSKV | SEQ ID NO: 53 |
| betacas-44 | VENLHLPLPLLQSWMHQPHQ | SEQ ID NO: 54 |
| betacas-48 | SWMHQPHQPLPPTVMFPPQS | SEQ ID NO: 55 |
| betacas-56 | SQSKVLPVPQKAVPYPQRDM | SEQ ID NO: 56 |
| betalac-18 | GDLEILLQKWENDECAQKKI | SEQ ID NO: 57 |
| betalac-44 | DEALEKFDKALKALPMHIRL | SEQ ID NO: 58 |
| kappacas-06 | RFFSDKIAKYIPIQYVLSRY | SEQ ID NO: 59 |
| kappacas-16 | KPVALINNQFLPYPYYAKPA | SEQ ID NO: 60 |
| kappacas-21 | YAKPAAVRSPAQILQWQVLS | SEQ ID NO: 61 |
| kappacas-24 | PAQILQWQVLSNTVPAKSCQ | SEQ ID NO: 62 |
| kappacas-30 | CQAQPTTMARHPHPHLSFMA | SEQ ID NO: 63 |
| kappacas-33 | RHPHPHLSFMAIPPKKNQDK | SEQ ID NO: 64 |
| kappacas-41 | TINTIASGEPTSTPTTEAVE | SEQ ID NO: 65 |
| kappacas-50 | DSPEVIESPPEINTVQVTST | SEQ ID NO: 66 |
| kappacas-51 | PEVIESPPEINTVQVTSTAV | SEQ ID NO: 67 |
| betalac-39 | QSLVCQCLVRTPEVDDEALE | SEQ ID NO: 68 |

### Examples

Sera were obtained from CM tolerant and CMA individuals and assayed in the LUMINEX assay to obtain the representative results shown in FIGS. 1-3. Wash buffer, sera, beads and antibody dilutions were prepared according to the manufacturer's directions. The filter plate was pre-wet with buffer for 1 min, and the buffer was removed by vacuum. 100 μl of the bead cocktail was added to each well, the buffer was removed by vacuum, and the beads were washed twice with 100 μl buffer. 100 μl of sera dilution was added to each well and incubated for 2 hrs. with shaking. Vacuum was applied to remove liquid. Beads were again washed twice with buffer. 50 μl of the antibody dilution was applied to each well and incubated for 30 min. with shaking. After application of vacuum the well was washed three times. 100 μl of buffer was added to the wells and the samples were transferred to a fixed plate. The wells were read on the LUMINEX instrument. Results are shown in FIGS. 1-3 and discussed above.

### Additional Applications of the Methods

The concepts of the invention with respect to allergenic epitope-containing peptides derived from cow's milk and their use for diagnosis of CMA, for detecting development of clinical tolerance to cow's milk proteins, and for detecting increases and decreases in the intensity of the allergy can also be applied to development of other allergenic epitope-containing peptide panels and their use in diagnosing, detecting tolerance, and detecting increases and development of tolerance to other allergenic proteins.

For example, allergenic epitope-containing peptide panels derived from allergenic peanut proteins, particularly the Ara h protein family, may be utilized in methods similar to those discussed above. Such a panel may include one or more peptides from the following list (SEQ ID NOs: 69-277):

| | | |
|---|---|---|
| ara h 1.006 | VLASVSATHAKSSPY | SEQ ID NO: 69 |
| ara h 1.007 | SVSATHAKSSPYQKK | SEQ ID NO: 70 |
| ara h 1.008 | ATHAKSSPYQKKTEN | SEQ ID NO: 71 |
| ara h 1.012 | TENPCAQRCLQSCQQ | SEQ ID NO: 72 |
| ara h 1.013 | PCAQRCLQSCQQEPD | SEQ ID NO: 73 |
| ara h 1.015 | LQSCQQEPDDLKQKA | SEQ ID NO: 74 |
| ara h 1.016 | CQQEPDDLKQKACES | SEQ ID NO: 75 |
| ara h 1.017 | EPDDLKQKACESRCT | SEQ ID NO: 76 |
| ara h 1.019 | QKACESRCTKLEYDP | SEQ ID NO: 77 |
| ara h 1.020 | CESRCTKLEYDPRCV | SEQ ID NO: 78 |
| ara h 1.021 | RCTKLEYDPRCVYDP | SEQ ID NO: 79 |
| ara h 1.022 | KLEYDPRCVYDPRGH | SEQ ID NO: 80 |
| ara h 1.023 | YDPRCVYDPRGHTGT | SEQ ID NO: 81 |
| ara h 1.025 | YDPRGHTGTTNQRSP | SEQ ID NO: 82 |
| ara h 1.026 | RGHTGTTNQRSPPGE | SEQ ID NO: 83 |
| ara h 1.028 | TNQRSPPGERTRGRQ | SEQ ID NO: 84 |
| ara h 1.029 | RSPPGERTRGRQPGD | SEQ ID NO: 85 |
| ara h 1.030 | PGERTRGRQPGDYDD | SEQ ID NO: 86 |
| ara h 1.031 | RTRGRQPGDYDDDRR | SEQ ID NO: 87 |
| ara h 1.032 | GRQPGDYDDDRRQPR | SEQ ID NO: 88 |
| ara h 1.033 | PGDYDDDRRQPRREE | SEQ ID NO: 89 |
| ara h 1.034 | YDDDRRQPRREEGGR | SEQ ID NO: 90 |
| ara h 1.035 | DRRQPRREEGGRWGP | SEQ ID NO: 91 |
| ara h 1.036 | QPRREEGGRWGPAGP | SEQ ID NO: 92 |
| ara h 1.037 | REEGGRWGPAGPRER | SEQ ID NO: 93 |
| ara h 1.038 | GGRWGPAGPRERERE | SEQ ID NO: 94 |
| ara h 1.039 | WGPAGPREREREEDW | SEQ ID NO: 95 |
| ara h 1.040 | AGPREREREEDWRQP | SEQ ID NO: 96 |
| ara h 1.041 | REREREEDWRQPRED | SEQ ID NO: 97 |
| ara h 1.042 | EREEDWRQPREDWRR | SEQ ID NO: 98 |
| ara h 1.043 | EDWRQPREDWRRPSH | SEQ ID NO: 99 |
| ara h 1.044 | RQPREDWRRPSHQQP | SEQ ID NO: 100 |
| ara h 1.045 | REDWRRPSHQQPRKI | SEQ ID NO: 101 |
| ara h 1.046 | WRRPSHQQPRKIRPE | SEQ ID NO: 102 |
| ara h 1.047 | PSHQQPRIGRPEGRE | SEQ ID NO: 103 |
| ara h 1.048 | QQPRKIRPEGREGEQ | SEQ ID NO: 104 |
| ara h 1.049 | RK1RPEGREGEQEWG | SEQ ID NO: 105 |
| ara h 1.050 | RPEGREGEQEWGTPG | SEQ ID NO: 106 |
| ara h 1.051 | GREGEQEWGTPGSHV | SEQ ID NO: 107 |
| ara h 1.052 | GEQEWGTPGSHVREE | SEQ ID NO: 108 |
| ara h 1.053 | EWGTPGSHVREETSR | SEQ ID NO: 109 |
| ara h 1.056 | REETSRNNPFYFPSR | SEQ ID NO: 110 |
| ara h 1.057 | TSRNNPFYFPSRRFS | SEQ ID NO: 111 |
| ara h 1.058 | NNPFYFPSRRFSTRY | SEQ ID NO: 112 |
| ara h 1.089 | RIPSGFISYILNRHD | SEQ ID NO: 113 |
| ara h 1.090 | SGFISYILNRHDNQN | SEQ ID NO: 114 |
| ara h 1.094 | NQNLRVAKISMPVNT | SEQ ID NO: 115 |
| ara h 1.095 | LRVAKISMPVNTPGQ | SEQ ID NO: 116 |
| ara h 1.096 | AKISMPVNTPGQFED | SEQ ID NO: 117 |
| ara h 1.097 | SMPVNTPGQFEDFFP | SEQ ID NO: 118 |
| ara h 1.098 | VNTPGQFEDFFPASS | SEQ ID NO: 119 |
| ara h 1.099 | PGQFEDFFPASSRDQ | SEQ ID NO: 120 |
| ara h 1.100 | FEDFFPASSRDQSSY | SEQ ID NO: 121 |
| ara h 1.102 | ASSRDQSSYLQGFSR | SEQ ID NO: 122 |
| ara h 1.103 | RDQSSYLQGFSRNTL | SEQ ID NO: 123 |
| ara h 1.104 | SSYLQGFSRNTLEAA | SEQ ID NO: 124 |
| ara h 1.105 | LQGFSRNTLEAAFNA | SEQ ID NO: 125 |
| ara h 1.112 | RVLLEENAGGEQEER | SEQ ID NO: 126 |
| ara h 1.113 | LEENAGGEQEERGQR | SEQ ID NO: 127 |
| ara h 1.114 | NAGGEQEERGQRRWS | SEQ ID NO: 128 |
| ara h 1.115 | GEQEERGQRRWSTRS | SEQ ID NO: 129 |
| ara h 1.116 | EERGQRRWSTRSSEN | SEQ ID NO: 130 |
| ara h 1.129 | KKGSEEEGDITNPIN | SEQ ID NO: 131 |
| ara h 1.130 | SEEEGDITNPINLRE | SEQ ID NO: 132 |
| ara h 1.131 | EGDITNPINLREGEP | SEQ ID NO: 133 |
| ara h 1.132 | ITNPINLREGEPDLS | SEQ ID NO: 134 |
| ara h 1.133 | PINLREGEPDLSNNF | SEQ ID NO: 135 |
| ara h 1.134 | LREGEPDLSNNFGKL | SEQ ID NO: 136 |
| ara h 1.135 | GEPDLSNNFGKLFEV | SEQ ID NO: 137 |
| ara h 1.136 | DLSNNFGKLFEVKPD | SEQ ID NO: 138 |
| ara h 1.137 | NNFGKLFEVKPDKKN | SEQ ID NO: 139 |
| ara h 1.138 | GKLFEVKPDKKNPQL | SEQ ID NO: 140 |
| ara h 1.141 | KKNPQLQDLDMMLTC | SEQ ID NO: 141 |
| ara h 1.142 | PQLQDLDMMLTCVEI | SEQ ID NO: 142 |
| ara h 1.143 | QDLDMMLTCVEIKEG | SEQ ID NO: 143 |
| ara h 1.144 | DMMLTCVEIKEGALM | SEQ ID NO: 144 |
| ara h 1.145 | LTCVEIKEGALMLPH | SEQ ID NO: 145 |
| ara h 1.146 | VEIKEGALMLPHFNS | SEQ ID NO: 146 |
| ara h 1.147 | KEGALMLPHFNSKAM | SEQ ID NO: 147 |
| ara h 1.165 | SNREVRRYTARLKEG | SEQ ID NO: 148 |
| ara h 1.166 | EVRRYTARLKEGDVF | SEQ ID NO: 149 |
| ara h 1.167 | RYTARLKEGDVFIMP | SEQ ID NO: 150 |
| ara h 1.168 | ARLKEGDVFIMPAAH | SEQ ID NO: 151 |
| ara h 1.169 | KEGDVFIMPAAHPVA | SEQ ID NO: 152 |
| ara h 1.170 | DVFIMPAAHPVAINA | SEQ ID NO: 153 |
| ara h 1.173 | PVAINASSELHLLGF | SEQ ID NO: 154 |
| ara h 1.174 | INASSELHLLGFGIN | SEQ ID NO: 155 |
| ara h 1.175 | SSELHLLGFGINAEN | SEQ ID NO: 156 |
| ara h 1.176 | LHLLGFGINAENNHR | SEQ ID NO: 157 |
| ara h 1.177 | LGFGINAENNHRIFL | SEQ ID NO: 158 |
| ara h 1.178 | GINAENNHRIFLAGD | SEQ ID NO: 159 |
| ara h 1.179 | AENNHRIFLAGDKDN | SEQ ID NO: 160 |
| ara h 1.180 | NHRIFLAGDKDNVID | SEQ ID NO: 161 |
| ara h 1.181 | IFLAGDKDNVIDQIE | SEQ ID NO: 162 |
| ara h 1.182 | AGDKDNVIDQIEKQA | SEQ ID NO: 163 |
| ara h 1.183 | KDNVIDQIEKQAKDL | SEQ ID NO: 164 |
| ara h 1.184 | VIDQIEKQAKDLAFP | SEQ ID NO: 165 |
| ara h 1.185 | QIEKQAKDLAFPGSG | SEQ ID NO: 166 |
| ara h 1.186 | KQAKDLAFPGSGEQV | SEQ ID NO: 167 |
| ara h 1.187 | KDLAFPGSGEQVEKL | SEQ ID NO: 168 |
| ara h 1.188 | AFPGSGEQVEKLIKN | SEQ ID NO: 169 |
| ara h 1.189 | GSGEQVEKLIKNQKE | SEQ ID NO: 170 |
| ara h 1.190 | EQVEKLIKNQKESHF | SEQ ID NO: 171 |
| ara h 1.191 | EKLIKNQKESHFVSA | SEQ ID NO: 172 |
| ara h 1.192 | IKNQKESHFVSARPQ | SEQ ID NO: 173 |
| ara h 1.193 | QKESHFVSARPQSQS | SEQ ID NO: 174 |
| ara h 1.194 | SHFVSARPQSQSQSP | SEQ ID NO: 175 |
| ara h 1.195 | VSARPQSQSQSPSSP | SEQ ID NO: 176 |
| ara h 1.196 | RPQSQSQSPSSPEKE | SEQ ID NO: 177 |
| ara h 1.197 | SQSQSPSSPEKESPE | SEQ ID NO: 178 |
| ara h 1.198 | QSPSSPEKESPEKED | SEQ ID NO: 179 |
| ara h 1.199 | SSPEKESPEKEDQEE | SEQ ID NO: 180 |
| ara h 1.200 | EKESPEKEDQEEENQ | SEQ ID NO: 181 |
| ara h 1.201 | SPEKEDQEEENQGGK | SEQ ID NO: 182 |
| ara h 1.202 | KEDQEEENQGGKGPL | SEQ ID NO: 183 |
| ara h 1.203 | QEEENQGGKGPLLSI | SEQ ID NO: 184 |
| ara h 2.005 | AAHASARQQWELQGD | SEQ ID NO: 185 |
| ara h 2.006 | ASARQQWELQGDRRC | SEQ ID NO: 186 |
| ara h 2.007 | RQQWELQGDRRCQSQ | SEQ ID NO: 187 |
| ara h 2.008 | WELQGDRRCQSQLER | SEQ ID NO: 188 |
| ara h 2.009 | QGDRRCQSQLERANL | SEQ ID NO: 189 |
| ara h 2.010 | RRCQSQLERANLRPC | SEQ ID NO: 190 |
| ara h 2.011 | QSQLERANLRPCEQH | SEQ ID NO: 191 |
| ara h 2.012 | LERANLRPCEQHLMQ | SEQ ID NO: 192 |
| ara h 2.013 | ANLRPCEQHLMQKIQ | SEQ ID NO: 193 |
| ara h 2.014 | RPCEQHLMQKIQRDE | SEQ ID NO: 194 |
| ara h 2.015 | EQHLMQKIQRDEDSY | SEQ ID NO: 195 |
| ara h 2.016 | LMQKIQRDEDSYERD | SEQ ID NO: 196 |
| ara h 2.017 | KIQRDEDSYERDPYS | SEQ ID NO: 197 |
| ara h 2.018 | RDEDSYERDPYSPSQ | SEQ ID NO: 198 |
| ara h 2.019 | DSYERDPYSPSQDPY | SEQ ID NO: 199 |
| ara h 2.020 | ERDPYSPSQDPYSPS | SEQ ID NO: 200 |
| ara h 2.021 | PYSPSQDPYSPSPYD | SEQ ID NO: 201 |
| ara h 2.022 | PSQDPYSPSPYDRRG | SEQ ID NO: 202 |
| ara h 2.023 | DPYSPSPYDRRGAGS | SEQ ID NO: 203 |
| ara h 2.024 | SPSPYDRRGAGSSQH | SEQ ID NO: 204 |
| ara h 2.025 | PYDRRGAGSSQHQER | SEQ ID NO: 205 |
| ara h 2.029 | QERCCNELNEFENNQ | SEQ ID NO: 206 |
| ara h 2.030 | CCNELNEFENNQRCM | SEQ ID NO: 207 |
| ara h 2.031 | ELNEFENNQRCMCEA | SEQ ID NO: 208 |
| ara h 2.032 | EFENNQRCMCEALQQ | SEQ ID NO: 209 |
| ara h 2.034 | RCMCEALQQIMENQS | SEQ ID NO: 210 |
| ara h 2.035 | CEALQQIMENQSDRL | SEQ ID NO: 211 |
| ara h 2.036 | LQQIMENQSDRLQGR | SEQ ID NO: 212 |
| ara h 2.037 | IMENQSDRLQGRQQE | SEQ ID NO: 213 |
| ara h 2.038 | NQSDRLQGRQQEQQF | SEQ ID NO: 214 |
| ara h 2.039 | DRLQGRQQEQQFKRE | SEQ ID NO: 215 |
| ara h 2.040 | QGRQQEQQFKRELRN | SEQ ID NO: 216 |
| ara h 2.041 | QQEQQFKRELRNLPQ | SEQ ID NO: 217 |
| ara h 2.042 | QQFKRELRNLPQQCG | SEQ ID NO: 218 |
| ara h 2.043 | KRELRNLPQQCGLRA | SEQ ID NO: 219 |
| ara h 2.045 | LPQQCGLRAPQRCDL | SEQ ID NO: 220 |
| ara h 2.046 | QCGLRAPQRCDLDVE | SEQ ID NO: 221 |
| ara h 2.047 | LRAPQRCDLDVESGG | SEQ ID NO: 222 |
| ara h 3.008 | RIESEGGYIETWNPN | SEQ ID NO: 223 |
| ara h 3.013 | NQEFECAGVALSRLV | SEQ ID NO: 224 |
| ara h 3.015 | AGVALSRLVLRRNAL | SEQ ID NO: 225 |
| ara h 3.016 | ALSRLVLRRNALRRP | SEQ ID NO: 226 |
| ara h 3.017 | RLVLRRNALRRPFYS | SEQ ID NO: 227 |
| ara h 3.018 | LRRNALRRPFYSNAP | SEQ ID NO: 228 |
| ara h 3.019 | NALRRPFYSNAPQEI | SEQ ID NO: 229 |
| ara h 3.030 | HYEEPHTQGRRSQSQ | SEQ ID NO: 230 |
| ara h 3.031 | EPHTQGRRSQSQRPP | SEQ ID NO: 231 |
| ara h 3.032 | TQGRRSQSQRPPRRL | SEQ ID NO: 232 |
| ara h 3.033 | RRSQSQRPPRRLQGE | SEQ ID NO: 233 |
| ara h 3.036 | RRLQGEDQSQQQRDS | SEQ ID NO: 234 |
| ara h 3.037 | QGEDQSQQQRDSHQK | SEQ ID NO: 235 |
| ara h 3.060 | NTEQEFLRYQQQSRQ | SEQ ID NO: 236 |
| ara h 3.061 | QEFLRYQQQSRQSRR | SEQ ID NO: 237 |
| ara h 3.068 | PYSPQSQPRQEEREF | SEQ ID NO: 238 |
| ara h 3.069 | PQSQPRQEEREFSPR | SEQ ID NO: 239 |
| ara h 3.070 | QPRQEEREFSPRGQH | SEQ ID NO: 240 |
| ara h 3.071 | QEEREFSPRGQHSRR | SEQ ID NO: 241 |
| ara h 3.073 | SPRGQHSRRERAGQE | SEQ ID NO: 242 |
| ara h 3.074 | GQHSRRERAGQEEEN | SEQ ID NO: 243 |
| ara h 3.075 | SRRERAGQEEENEGG | SEQ ID NO: 244 |
| ara h 3.077 | GQEEENEGGNIFSGF | SEQ ID NO: 245 |
| ara h 3.078 | EENEGGNIFSGFTPE | SEQ ID NO: 246 |
| ara h 3.079 | EGGNIFSGFTPEFLE | SEQ ID NO: 247 |
| ara h 3.080 | NIFSGFTPEFLEQAF | SEQ ID NO: 248 |
| ara h 3.081 | SGFTPEFLEQAFQVD | SEQ ID NO: 249 |
| ara h 3.082 | TPEFLEQAFQVDDRQ | SEQ ID NO: 250 |
| ara h 3.090 | ESEEEGAIVTVRGGL | SEQ ID NO: 251 |
| ara h 3.091 | EEGAIVTVRGGLRIL | SEQ ID NO: 252 |
| ara h 3.092 | AIVTVRGGLRILSPD | SEQ ID NO: 253 |
| ara h 3.093 | TVRGGLRILSPDRKR | SEQ ID NO: 254 |
| ara h 3.094 | GGLRILSPDRKRRAD | SEQ ID NO: 255 |
| ara h 3.095 | RILSPDRKRRADEEE | SEQ ID NO: 256 |
| ara h 3.097 | RKRRADEEEEYDEDE | SEQ ID NO: 257 |
| ara h 3.098 | RADEEEEYDEDEYEY | SEQ ID NO: 258 |
| ara h 3.099 | EEEEYDEDEYEYDEE | SEQ ID NO: 259 |
| ara h 3.100 | EYDEDEYEYDEEDRR | SEQ ID NO: 260 |
| ara h 3.101 | EDEYEYDEEDRRRGR | SEQ ID NO: 261 |
| ara h 3.102 | YEYDEEDRRRGRGSR | SEQ ID NO: 262 |
| ara h 3.103 | DEEDRRRGRGSRGRG | SEQ ID NO: 263 |
| ara h 3.104 | DRRRGRGSRGRGNGI | SEQ ID NO: 264 |
| ara h 3.105 | RGRGSRGRGNGIEET | SEQ ID NO: 265 |
| ara h 3.106 | GSRGRGNGIEETICT | SEQ ID NO: 266 |
| ara h 3.107 | GRGNGIEETICTASA | SEQ ID NO: 267 |
| ara h 3.108 | NGIEETICTASAKKN | SEQ ID NO: 268 |
| ara h 3.152 | IANLAGENSVIDNLP | SEQ ID NO: 269 |
| ara h 3.153 | LAGENSVIDNLPEEV | SEQ ID NO: 270 |
| ara h 3.154 | ENSVIDNLPEEVVAN | SEQ ID NO: 271 |
| ara h 3.155 | VIDNLPEEVVANSYG | SEQ ID NO: 272 |
| ara h 3.161 | EQARQLKNNNPFKFF | SEQ ID NO: 273 |
| ara h 3.162 | RQLKNNNPFKFFVPP | SEQ ID NO: 274 |
| ara h 3.163 | KNNNPFKFFVPPSQQ | SEQ ID NO: 275 |
| ara h 3.164 | NPFKFFVPPSQQSPR | SEQ ID NO: 276 |
| ara h 3.165 | KFFVPPSQQSPRAVA | SEQ ID NO: 277 |

In a further example, allergenic epitope-containing peptide panels derived from allergenic egg proteins, particularly ovalbumin (ova) and/or ovomucoid (ovm), may be utilized in methods similar to those discussed above. Such a panel may include one or more peptides from the following list (SEQ ID NOs: 278-460):

| | | |
|---|---|---|
| ova-1 | MGSIGAASMEFCFDV | SEQ ID NO: 278 |
| ova-2 | IGAASMEFCFDVFKE | SEQ ID NO: 279 |
| ova-3 | ASMEFCFDVFKELKV | SEQ ID NO: 280 |
| ova-4 | EFCFDVFKELKVHHA | SEQ ID NO: 281 |
| ova-5 | FDVFKELKVHHANEN | SEQ ID NO: 282 |
| ova-6 | FKELKVHHANENIFY | SEQ ID NO: 283 |
| ova-7 | LKVHHANENIFYCPI | SEQ ID NO: 284 |
| ova-8 | HHANENIFYCPIAIM | SEQ ID NO: 285 |
| ova-9 | NENIFYCPIAIMSAL | SEQ ID NO: 286 |
| ova10 | IFYCPIAIMSALAMV | SEQ ID NO: 287 |
| ova-11 | CPIAIMSALAMVYLG | SEQ ID NO: 288 |
| ova-12 | AIMSALAMVYLGAKD | SEQ ID NO: 289 |
| ova-13 | SALAMVYLGAKDSTR | SEQ ID NO: 290 |
| ova-14 | AMVYLGAKDSTRTQI | SEQ ID NO: 291 |
| ova-15 | YLGAKDSTRTQINKV | SEQ ID NO: 292 |
| ova-16 | AKDSTRTQINKVVRF | SEQ ID NO: 293 |
| ova-17 | STRTQINKVVRFDKL | SEQ ID NO: 294 |
| ova-18 | TQINKVVRFDKLPGF | SEQ ID NO: 295 |
| ova-19 | NKVVRFDKLPGFGDS | SEQ ID NO: 296 |
| ova-20 | VRFDKLPGFGDSIEA | SEQ ID NO: 297 |
| ova-21 | DKLPGFGDSIEAQCG | SEQ ID NO: 298 |
| ova-22 | PGFGDSIEAQCGTSV | SEQ ID NO: 299 |
| ova-23 | GDSIEAQCGTSVNVH | SEQ ID NO: 300 |
| ova-24 | IEAQCGTSVNVHSSL | SEQ ID NO: 301 |
| ova-25 | QCGTSVNVHSSLRDI | SEQ ID NO: 302 |
| ova-26 | TSVNVHSSLRDILNQ | SEQ ID NO: 303 |
| ova-27 | NVHSSLRDILNQITK | SEQ ID NO: 304 |
| ova-28 | SSLRDILNQITKPND | SEQ ID NO: 305 |
| ova-29 | RDILNQITKPNDVYS | SEQ ID NO: 306 |
| ova-30 | LNQITKPNDVYSFSL | SEQ ID NO: 307 |
| ova-31 | ITKPNDVYSFSLASR | SEQ ID NO: 308 |
| ova-32 | PNDVYSFSLASRLYA | SEQ ID NO: 309 |
| ova-33 | VYSFSLASRLYAEER | SEQ ID NO: 310 |
| ova-34 | FSLASRLYAEERYPI | SEQ ID NO: 311 |
| ova-35 | ASRLYAEERYPILPE | SEQ ID NO: 312 |
| ova-36 | LYAEERYPILPEYLQ | SEQ ID NO: 313 |
| ova-37 | EERYPILPEYLQCVK | SEQ ID NO: 314 |
| ova-38 | YPILPEYLQCVKELY | SEQ ID NO: 315 |
| ova-39 | LPEYLQCVKELYRGG | SEQ ID NO: 316 |
| ova-40 | YLQCVKELYRGGLEP | SEQ ID NO: 317 |
| ova-41 | CVKELYRGGLEPINF | SEQ ID NO: 318 |
| ova-42 | ELYRGGLEPINFQTA | SEQ ID NO: 319 |
| ova-43 | RGGLEPINFQTAADQ | SEQ ID NO: 320 |
| ova-44 | LEPINFQTAADQARE | SEQ ID NO: 321 |
| ova-45 | INFQTAADQARELIN | SEQ ID NO: 322 |
| ova-46 | QTAADQARELINSWV | SEQ ID NO: 323 |
| ova-47 | ADQARELINSWVESQ | SEQ ID NO: 324 |
| ova-48 | ARELINSWVESQTNG | SEQ ID NO: 325 |
| ova-49 | LINSWVESQTNGIIR | SEQ ID NO: 326 |
| ova-50 | SWVESQTNGIIRNVL | SEQ ID NO: 327 |
| ova-51 | ESQTNGIIRNVLQPS | SEQ ID NO: 328 |
| ova-52 | TNGIIRNVLQPSSVD | SEQ ID NO: 329 |
| ova-53 | IIRNVLQPSSVDSQT | SEQ ID NO: 330 |
| ova-54 | NVLQPSSVDSQTAMV | SEQ ID NO: 331 |
| ova-55 | QPSSVDSQTAMVLVN | SEQ ID NO: 332 |
| ova-56 | SVDSQTAMVLVNAIV | SEQ ID NO: 333 |
| ova-57 | SQTAMVLVNAIVFKG | SEQ ID NO: 334 |
| ova-58 | AMVLVNAIVFKGLWE | SEQ ID NO: 335 |
| ova-59 | LVNAIVFKGLWEKAF | SEQ ID NO: 336 |
| ova-60 | AIVFKGLWEKAFKDE | SEQ ID NO: 337 |
| ova-61 | FKGLWEKAFKDEDTQ | SEQ ID NO: 338 |
| ova-62 | LWEKAFKDEDTQAMP | SEQ ID NO: 339 |
| ova-63 | KAFKDEDTQAMPFRV | SEQ ID NO: 340 |
| ova-64 | KDEDTQAMPFRVTEQ | SEQ ID NO: 341 |
| ova-65 | DTQAMPFRVTEQESK | SEQ ID NO: 342 |
| ova-66 | AMPFRVTEQESKPVQ | SEQ ID NO: 343 |
| ova-67 | FRVTEQESKPVQMMY | SEQ ID NO: 344 |
| ova-68 | TEQESKPVQMMYQIG | SEQ ID NO: 345 |
| ova-69 | ESKPVQMMYQIGLFR | SEQ ID NO: 346 |
| ova-70 | PVQMMYQIGLFRVAS | SEQ ID NO: 347 |
| ova-71 | MMYQIGLFRVASMAS | SEQ ID NO: 348 |
| ova-72 | QIGLFRVASMASEKM | SEQ ID NO: 349 |
| ova-73 | LFRVASMASEKMKIL | SEQ ID NO: 350 |
| ova-74 | VASMASEKMKILELP | SEQ ID NO: 351 |
| ova-75 | MASEKMKILELPFAS | SEQ ID NO: 352 |
| ova-76 | EKMKILELPFASGTM | SEQ ID NO: 353 |
| ova-77 | KILELPFASGTMSML | SEQ ID NO: 354 |
| ova-78 | ELPFASGTMSMLVLL | SEQ ID NO: 355 |
| ova-79 | FASGTMSMLVLLPDE | SEQ ID NO: 356 |
| ova-80 | GTMSMLVLLPDEVSG | SEQ ID NO: 357 |
| ova-81 | SMLVLLPDEVSGLEQ | SEQ ID NO: 358 |
| ova-82 | VLLPDEVSGLEQLES | SEQ ID NO: 359 |
| ova-83 | PDEVSGLEQLESIIN | SEQ ID NO: 360 |
| ova-84 | VSGLEQLESIINFEK | SEQ ID NO: 361 |
| ova-85 | LEQLESIINFEKLTE | SEQ ID NO: 362 |
| ova-86 | LESIINFEKLTEWTS | SEQ ID NO: 363 |
| ova-87 | IINFEKLTEWTSSNV | SEQ ID NO: 364 |
| ova-88 | FEKLTEWTSSNVMEE | SEQ ID NO: 365 |
| ova-89 | LTEWTSSNVMEERKI | SEQ ID NO: 366 |
| ova-90 | WTSSNVMEERKIKVY | SEQ ID NO: 367 |
| ova-91 | SNVMEERKIKVYLPR | SEQ ID NO: 368 |
| ova-92 | MEERKIKVYLPRMKM | SEQ ID NO: 369 |
| ova-93 | RKIKVYLPRMKMEEK | SEQ ID NO: 370 |
| ova-94 | KVYLPRMKMEEKYNL | SEQ ID NO: 371 |
| ova-95 | LPRMKMEEKYNLTSV | SEQ ID NO: 372 |
| ova-96 | MKMEEKYNLTSVLMA | SEQ ID NO: 373 |
| ova-97 | EEKYNLTSVLMAMGI | SEQ ID NO: 374 |
| ova-98 | YNLTSVLMAMGITDV | SEQ ID NO: 375 |
| ova-99 | TSVLMAMGITDVFSS | SEQ ID NO: 376 |
| ova-100 | LMAMGITDVFSSSAN | SEQ ID NO: 377 |
| ova-101 | MGITDVFSSSANLSG | SEQ ID NO: 378 |
| ova-102 | TDVFSSSANLSGISS | SEQ ID NO: 379 |
| ova-103 | FSSSANLSGISSAES | SEQ ID NO: 380 |
| ova-104 | SANLSGISSAESLKI | SEQ ID NO: 381 |
| ova-105 | LSGISSAESLKISQA | SEQ ID NO: 382 |
| ova-106 | ISSAESLKISQAVHA | SEQ ID NO: 383 |
| ova-107 | AESLKISQAVHAAHA | SEQ ID NO: 384 |
| ova-108 | LKISQAVHAAHAEIN | SEQ ID NO: 385 |
| ova-109 | SQAVHAAHAEINEAG | SEQ ID NO: 386 |
| ova-110 | VHAAHAEINEAGREV | SEQ ID NO: 387 |
| ova-111 | AHAEINEAGREVVGS | SEQ ID NO: 388 |
| ova-112 | EINEAGREVVGSAEA | SEQ ID NO: 389 |
| ova-113 | EAGREVVGSAEAGVD | SEQ ID NO: 390 |
| ova-114 | REVVGSAEAGVDAAS | SEQ ID NO: 391 |
| ova-115 | VGSAEAGVDAASVSE | SEQ ID NO: 392 |
| ova-116 | AEAGVDAASVSEEFR | SEQ ID NO: 393 |
| ova-117 | GVDAASVSEEFRADH | SEQ ID NO: 394 |
| ova-118 | AASVSEEFRADHPFL | SEQ ID NO: 395 |
| ova-119 | VSEEFRADHPFLFCI | SEQ ID NO: 396 |
| ova-120 | EFRADHPFLFCIKHI | SEQ ID NO: 397 |
| ova-121 | ADHPFLFCIKHIATN | SEQ ID NO: 398 |
| ova-122 | PFLFCIKHIATNAVL | SEQ ID NO: 399 |
| ova-123 | FCIKHIATNAVLFFG | SEQ ID NO: 400 |
| ova-124 | KHIATNAVLFFGRCV | SEQ ID NO: 401 |
| ova-125 | ATNAVLFFGRCVSP | SEQ ID NO: 402 |
| ovm-1 | AEVDCSRFPNATDKE | SEQ ID NO: 403 |
| ovm-2 | DCSRFPNATDKEGKD | SEQ ID NO: 404 |
| ovm-3 | RFPNATDKEGKDVLV | SEQ ID NO: 405 |
| ovm-4 | NATDKEGKDVLVCNK | SEQ ID NO: 406 |
| ovm-5 | DKEGKDVLVCNKDLR | SEQ ID NO: 407 |
| ovm-6 | GKDVLVCNKDLRPIC | SEQ ID NO: 408 |
| ovm-7 | VLVCNKDLRPICGTD | SEQ ID NO: 409 |
| ovm-8 | CNKDLRPICGTDGVT | SEQ ID NO: 410 |
| ovm-9 | DLRPICGTDGVTYTN | SEQ ID NO: 411 |
| ovm-10 | PICGTDGVTYTNDCL | SEQ ID NO: 412 |
| ovm-11 | GTDGVTYTNDCLLCA | SEQ ID NO: 413 |
| ovm-12 | GVTYTNDCLLCAYSI | SEQ ID NO: 414 |
| ovm-13 | YTNDCLLCAYSIEFG | SEQ ID NO: 415 |
| ovm-14 | DCLLCAYSIEFGTNI | SEQ ID NO: 416 |
| ovm-15 | LCAYSIEFGTNISKE | SEQ ID NO: 417 |
| ovm-16 | YSIEFGTNISKEHDG | SEQ ID NO: 418 |
| ovm-17 | EFGTNISKEHDGECK | SEQ ID NO: 419 |
| ovm-18 | TNISKEHDGECKETV | SEQ ID NO: 420 |
| ovm-19 | SKEHDGECKETVPMN | SEQ ID NO: 421 |
| ovm-20 | HDGECKETVPMNCSS | SEQ ID NO: 422 |
| ovm-21 | ECKETVPMNCSSYAN | SEQ ID NO: 423 |
| ovm-22 | ETVPMNCSSYANTTS | SEQ ID NO: 424 |
| ovm-23 | PMNCSSYANTTSEDG | SEQ ID NO: 425 |
| ovm-24 | CSSYANTTSEDGKVM | SEQ ID NO: 426 |
| ovm-25 | YANTTSEDGKVMVLC | SEQ ID NO: 427 |
| ovm-26 | TTSEDGKVMVLCNRA | SEQ ID NO: 428 |
| ovm-27 | EDGKVMVLCNRAFNP | SEQ ID NO: 429 |
| ovm-28 | KVMVLCNRAFNPVCG | SEQ ID NO: 430 |
| ovm-29 | VLCNRAFNPVCGTDG | SEQ ID NO: 431 |
| ovm-30 | NRAFNPVCGTDGVTY | SEQ ID NO: 432 |
| ovm-31 | FNPVCGTDGVTYDNE | SEQ ID NO: 433 |
| ovm-32 | VCGTDGVTYDNECLL | SEQ ID NO: 434 |
| ovm-33 | TDGVTYDNECLLCAH | SEQ ID NO: 435 |
| ovm-34 | VTYDNECLLCAHKVE | SEQ ID NO: 436 |
| ovm-35 | DNECLLCAHKVEQGA | SEQ ID NO: 437 |
| ovm-36 | CLLCAHKVEQGASVD | SEQ ID NO: 438 |
| ovm-37 | CAHKVEQGASVDKRH | SEQ ID NO: 439 |
| ovm-38 | KVEQGASVDKRHDGG | SEQ ID NO: 440 |
| ovm-39 | QGASVDKRHDGGCRK | SEQ ID NO: 441 |
| ovm-40 | SVDKRHDGGCRKELA | SEQ ID NO: 442 |
| ovm-41 | KRHDGGCRKELAAVS | SEQ ID NO: 443 |
| ovm-42 | DGGCRKELAAVSVDC | SEQ ID NO: 444 |
| ovm-43 | CRKELAAVSVDCSEY | SEQ ID NO: 445 |
| ovm-44 | ELAAVSVDCSEYPKP | SEQ ID NO: 446 |
| ovm-45 | AVSVDCSEYPKPDCT | SEQ ID NO: 447 |
| ovm-46 | VDCSEYPKPDCTAED | SEQ ID NO: 448 |
| ovm-47 | SEYPKPDCTAEDRPL | SEQ ID NO: 449 |
| ovm-48 | PKPDCTAEDRPLCGS | SEQ ID NO: 450 |
| ovm-49 | DCTAEDRPLCGSDNK | SEQ ID NO: 451 |
| ovm-50 | AEDRPLCGSDNKTYG | SEQ ID NO: 452 |
| ovm-51 | RPLCGSDNKTYGNKC | SEQ ID NO: 453 |
| ovm-52 | CGSDNKTYGNKCNFC | SEQ ID NO: 454 |
| ovm-53 | DNKTYGNKCNFCNAV | SEQ ID NO: 455 |
| ovm-54 | TYGNKCNFCNAVVES | SEQ ID NO: 456 |
| ovm-55 | NKCNFCNAVVESNGT | SEQ ID NO: 457 |
| ovm-56 | NFCNAVVESNGTLTL | SEQ ID NO: 458 |
| ovm-57 | NAVVESNGTLTLSHF | SEQ ID NO: 459 |
| ovm-58 | VESNGTLTLSHFGKC | SEQ ID NO: 460 |

In a further example, allergenic epitope-containing peptide panels derived from allergenic shrimp proteins, particularly arginine kinase (ak), myosin light chain (mlc), sarcoplasmic calcium binding protein (scp), tropomyosin (tm) and Troponin C (tpc), may be utilized in methods similar to those discussed above. Such a panel may include one or more peptides from the following list SEQ ID NOs: 461-683):

| | | |
|---|---|---|
| ak-01 | H-MADAAVIEKLEAGFK-OH | SEQ ID NO: 461 |
| ak-02 | H-VIEKLEAGFKKLEAA-OH | SEQ ID NO: 462 |
| ak-03 | H-EAGFKKLEAATDCKS-OH | SEQ ID NO: 463 |
| ak-04 | H-KLEAATDCKSLLKKY-OH | SEQ ID NO: 464 |
| ak-05 | H-TDCKSLLKKYLTKEV-OH | SEQ ID NO: 465 |
| ak-06 | H-LLKKYLTKEVFDKLK-OH | SEQ ID NO: 466 |
| ak-07 | H-LTKEVFDKLKDKKTS-OH | SEQ ID NO: 467 |
| ak-08 | H-FDKLKDKKTSLGATL-OH | SEQ ID NO: 468 |
| ak-09 | H-DKKTSLGATLLDVIQ-OH | SEQ ID NO: 469 |
| ak-10 | H-LGATLLDVIQSGVEN-OH | SEQ ID NO: 470 |
| ak-11 | H-LDVIQSGVENLDSGV-OH | SEQ ID NO: 471 |
| ak-12 | H-SGVENLDSGVGIYAP-OH | SEQ ID NO: 472 |
| ak-13 | H-LDSGVGIYAPDAEAY-OH | SEQ ID NO: 473 |
| ak-14 | H-GIYAPDAEAYTLFAP-OH | SEQ ID NO: 474 |
| ak-15 | H-DAEAYTLFAPLFDPI-OH | SEQ ID NO: 475 |
| ak-16 | H-TLFAPLFDPIIEDYH-OH | SEQ ID NO: 476 |
| ak-17 | H-LFDPIIEDYHVGFKQ-OH | SEQ ID NO: 477 |
| ak-18 | H-IEDYHVGFKQTDKHP-OH | SEQ ID NO: 478 |
| ak-19 | H-VGFKQTDKHPNKDFG-OH | SEQ ID NO: 479 |
| ak-20 | H-TDKHPNKDFGDVNSF-OH | SEQ ID NO: 480 |
| ak-21 | H-NKDFGDVNSFVNVDP-OH | SEQ ID NO: 481 |
| ak-22 | H-DVNSFVNVDPEGKFV-OH | SEQ ID NO: 482 |
| ak-23 | H-VNVDPEGKFVISTRV-OH | SEQ ID NO: 483 |
| ak-24 | H-EGKFVISTRVRCGRS-OH | SEQ ID NO: 484 |
| ak-25 | H-ISTRVRCGRSMQGYP-OH | SEQ ID NO: 485 |
| ak-26 | H-RCGRSMQGYPFNPCL-OH | SEQ ID NO: 486 |
| ak-27 | H-MQGYPFNPCLTESQY-OH | SEQ ID NO: 487 |
| ak-28 | H-FNPCLTESQYKEMEA-OH | SEQ ID NO: 488 |
| ak-29 | H-TESQYKEMEAKVSST-OH | SEQ ID NO: 489 |
| ak-30 | H-KEMEAKVSSTLSSLE-OH | SEQ ID NO: 490 |
| ak-31 | H-KVSSTLSSLEGELKG-OH | SEQ ID NO: 491 |
| ak-32 | H-LSSLEGELKGTYYPL-OH | SEQ ID NO: 492 |
| ak-33 | H-GELKGTYYPLTGMSK-OH | SEQ ID NO: 493 |
| ak-34 | H-TYYPLTGMSKEVQQK-OH | SEQ ID NO: 494 |
| ak-35 | H-TGMSKEVQQKLIDDH-OH | SEQ ID NO: 495 |
| ak-36 | H-EVQQKLIDDHFLFKE-OH | SEQ ID NO: 496 |
| ak-37 | H-LIDDHFLEKEGDRFL-OH | SEQ ID NO: 497 |
| ak-38 | H-FLEKEGDRELQAANA-OH | SEQ ID NO: 498 |
| ak-39 | H-GDRFLQAANACRYWP-OH | SEQ ID NO: 499 |
| ak-40 | H-QAANACRYWPAGRGI-OH | SEQ ID NO: 500 |
| ak-41 | H-CRYWPAGRGIYHNDN-OH | SEQ ID NO: 501 |
| ak-42 | H-AGRGIYHNDNKTFLV-OH | SEQ ID NO: 502 |
| ak-43 | H-YHNDNKTFLVWVNEE-OH | SEQ ID NO: 503 |
| ak-44 | H-KTFLVWVNEEDHLRI-OH | SEQ ID NO: 504 |
| ak-45 | H-WVNEEDHLRIISMQM-OH | SEQ ID NO: 505 |
| ak-46 | H-DHLRIISMQMGGDLG-OH | SEQ ID NO: 506 |
| ak-47 | H-ISMQMGGDLGQVFRR-OH | SEQ ID NO: 507 |
| ak-48 | H-GGDLGQVFRRLTSAV-OH | SEQ ID NO: 508 |
| ak-49 | H-QVFRRLTSAVNEIEK-OH | SEQ ID NO: 509 |
| ak-50 | H-LTSAVNEIEKRIPFS-OH | SEQ ID NO: 510 |
| ak-51 | H-NEIEKRIPFSHHDRL-OH | SEQ ID NO: 511 |
| ak-52 | H-RIPFSHHDRLGFLTF-OH | SEQ ID NO: 512 |
| ak-53 | H-HHDRLGFLTFCPTNL-OH | SEQ ID NO: 513 |
| ak-54 | H-GFLTFCPTNLGTTVR-OH | SEQ ID NO: 514 |
| ak-55 | H-CPTNLGTTVRASVHI-OH | SEQ ID NO: 515 |
| ak-56 | H-GTTVRASVHIKLPKL-OH | SEQ ID NO: 516 |
| ak-57 | H-ASVHIKLPKLAANRE-OH | SEQ ID NO: 517 |
| ak-58 | H-KLPKLAANREKLEEV-OH | SEQ ID NO: 518 |
| ak-59 | H-AANREKLEEVAGKYN-OH | SEQ ID NO: 519 |
| ak-60 | H-KLEEVAGKYNLQVRG-OH | SEQ ID NO: 520 |
| ak-61 | H-AGKYNLQVRGTRGEH-OH | SEQ ID NO: 521 |
| ak-62 | H-LQVRGTRGEHTEAEG-OH | SEQ ID NO: 522 |
| ak-63 | H-TRGEHTEAEGGIYDI-OH | SEQ ID NO: 523 |
| ak-64 | H-TEAEGGIYDISNKRR-OH | SEQ ID NO: 524 |
| ak-65 | H-GIYDISNKRRMGLTE-OH | SEQ ID NO: 525 |
| ak-66 | H-SNKRRMGLTEFQAVK-OH | SEQ ID NO: 526 |
| ak-67 | H-MGLTEFQAVKEMQDG-OH | SEQ ID NO: 527 |
| ak-68 | H-FQAVKEMQDGILELI-OH | SEQ ID NO: 528 |
| ak-69 | H-EMQDGILELIKIEKE-OH | SEQ ID NO: 529 |
| mlc-01 | H-MSRKSGSRSSSKRSK-OH | SEQ ID NO: 530 |
| mlc-02 | H-GSRSSSKRSKKSGGG-OH | SEQ ID NO: 531 |
| mlc-03 | H-SKRSKKSGGGSNVFD-OH | SEQ ID NO: 532 |
| mlc-04 | H-KSGGGSNVFDMFTQR-OH | SEQ ID NO: 533 |
| mlc-05 | H-SNVFDMFTQRQVAEF-OH | SEQ ID NO: 534 |
| mlc-06 | H-MFTQRQVAEFKEGFQ-OH | SEQ ID NO: 535 |
| mlc-07 | H-QVAEFKEGFQLMDRD-OH | SEQ ID NO: 536 |
| mlc-08 | H-KEGFQLMDRDKDGVI-OH | SEQ ID NO: 537 |
| mlc-09 | H-LMDRDKDGVIGKTDL-OH | SEQ ID NO: 538 |
| mlc-10 | H-KDGVIGKTDLRGTFD-OH | SEQ ID NO: 539 |
| mlc-11 | H-GKTDLRGTFDEIGRI-OH | SEQ ID NO: 540 |
| mlc-12 | H-RGTFDEIGRIATDQE-OH | SEQ ID NO: 541 |
| mlc-13 | H-EIGRIATDQELDEML-OH | SEQ ID NO: 542 |
| mlc-14 | H-ATDQELDEMLADAPA-OH | SEQ ID NO: 543 |
| mlc-15 | H-LDEMLADAPAPINFT-OH | SEQ ID NO: 544 |
| mlc-16 | H-ADAPAPINFTMLLNM-OH | SEQ ID NO: 545 |
| mlc-17 | H-PINFTMLLNMFAERQ-OH | SEQ ID NO: 546 |
| mlc-18 | H-MLLNMFAERQTGESD-OH | SEQ ID NO: 547 |
| mlc-19 | H-FAERQTGESDDDDVV-OH | SEQ ID NO: 548 |
| mlc-20 | H-TGESDDDDVVAKAFL-OH | SEQ ID NO: 549 |
| mlc-21 | H-DDDVVAKAFLAFADE-OH | SEQ ID NO: 550 |
| mlc-22 | H-AKAFLAFADEEGNID-OH | SEQ ID NO: 551 |
| mlc-23 | H-AFADEEGNIDCDTFR-OH | SEQ ID NO: 552 |
| mlc-24 | H-EGNIDCDTFRHALMT-OH | SEQ ID NO: 553 |
| mlc-25 | H-CDTFRHALMTWGDKF-OH | SEQ ID NO: 554 |
| mlc-26 | H-HALMTWGDKFSSQEA-OH | SEQ ID NO: 555 |
| mlc-27 | H-WGDKFSSQEADDALD-OH | SEQ ID NO: 556 |
| mlc-28 | H-SSQEADDALDQMDID-OH | SEQ ID NO: 557 |
| mlc-29 | H-DDALDQMDIDDGGKI-OH | SEQ ID NO: 558 |
| mlc-30 | H-QMDIDDGGKIDVQGV-OH | SEQ ID NO: 559 |
| mlc-31 | H-DGGKIDVQGVIQMLT-OH | SEQ ID NO: 560 |
| mlc-32 | H-DVQGVIQMLTAGGGD-OH | SEQ ID NO: 561 |
| mlc-33 | H-IQMLTAGGGDDAAAE-OH | SEQ ID NO: 562 |
| mlc-34 | H-AGGGDDAAAEEA-OH | SEQ ID NO: 563 |
| scp-01 | H-MAYSWDNRVKYVVRY-OH | SEQ ID NO: 564 |
| scp-02 | H-DNRVKYVVRYMYDID-OH | SEQ ID NO: 565 |
| scp-03 | H-YVVRYMYDIDNNGFL-OH | SEQ ID NO: 566 |
| scp-04 | H-MYDIDNNGFLDKNDF-OH | SEQ ID NO: 567 |
| scp-05 | H-NNGFLDKNDFECLAV-OH | SEQ ID NO: 568 |
| scp-06 | H-DKNDFECLAVRNTLI-OH | SEQ ID NO: 569 |
| scp-07 | H-ECLAVRNTLIEGRGE-OH | SEQ ID NO: 570 |
| scp-08 | H-RNTLIEGRGEFSADA-OH | SEQ ID NO: 571 |
| scp-09 | H-EGRGEFSADAYANNQ-OH | SEQ ID NO: 572 |
| scp-10 | H-FSADAYANNQKIMRN-OH | SEQ ID NO: 573 |
| scp-11 | H-YANNQKIMRNLWNEI-OH | SEQ ID NO: 574 |
| scp-12 | H-KIMRNLWNEIAELAD-OH | SEQ ID NO: 575 |
| scp-13 | H-LWNEIAELADFNKDG-OH | SEQ ID NO: 576 |
| scp-14 | H-AELADFNKDGEVTVD-OH | SEQ ID NO: 577 |
| scp-15 | H-FNKDGEVTVDEFKQA-OH | SEQ ID NO: 578 |
| scp-16 | H-EVTVDEFKQAVQKHC-OH | SEQ ID NO: 579 |
| scp-17 | H-EFKQAVQKHCQGKKY-OH | SEQ ID NO: 580 |
| scp-18 | H-VQKHCQGKKYGDFPG-OH | SEQ ID NO: 581 |
| scp-19 | H-QGKKYGDFPGAFKVF-OH | SEQ ID NO: 582 |
| scp-20 | H-GDFPGAFKVFIANQF-OH | SEQ ID NO: 583 |
| scp-21 | H-AFKVFIANQFKAIDV-OH | SEQ ID NO: 584 |
| scp-22 | H-IANQFKAIDVNGDGK-OH | SEQ ID NO: 585 |
| scp-23 | H-KAIDVNGDGKVGLDE-OH | SEQ ID NO: 586 |
| scp-24 | H-NGDGKVGLDEYRLDC-OH | SEQ ID NO: 587 |
| scp-25 | H-VGLDEYRLDCITRSA-OH | SEQ ID NO: 588 |
| scp-26 | H-YRLDCITRSAFAEVK-OH | SEQ ID NO: 589 |
| scp-27 | H-ITRSAFAEVKEIDDA-OH | SEQ ID NO: 590 |
| scp-28 | H-FAEVKEIDDAYNKLT-OH | SEQ ID NO: 591 |
| scp-29 | H-EIDDAYNKLTTEDDR-OH | SEQ ID NO: 592 |
| scp-30 | H-YNKLTTEDDRKAGGL-OH | SEQ ID NO: 593 |
| scp-31 | H-TEDDRKAGGLTLERY-OH | SEQ ID NO: 594 |
| scp-32 | H-KAGGLTLERYQDLYA-OH | SEQ ID NO: 595 |
| scp-33 | H-TLERYQDLYAQFISN-OH | SEQ ID NO: 596 |
| scp-34 | H-QDLYAQFISNPDESC-OH | SEQ ID NO: 597 |
| scp-35 | H-QFISNPDESCSACYL-OH | SEQ ID NO: 598 |
| scp-36 | H-PDESCSACYLFGPLK-OH | SEQ ID NO: 599 |
| scp-37 | H-SACYLFGPLKVVQ-OH | SEQ ID NO: 600 |
| tm-01 | H-MDAIKKKMQAMKLEK-OH | SEQ ID NO: 601 |
| tm-02 | H-KKMQAMKLEKDNAMD-OH | SEQ ID NO: 602 |
| tm-03 | H-MKLEKDNAMDRADTL-OH | SEQ ID NO: 603 |
| tm-04 | H-DNAMDRADTLEQQNK-OH | SEQ ID NO: 604 |
| tm-05 | H-RADTLEQQNKEANNR-OH | SEQ ID NO: 605 |
| tm-06 | H-EQQNKEANNRAEKSE-OH | SEQ ID NO: 606 |
| tm-07 | H-EANNRAEKSEEEVHN-OH | SEQ ID NO: 607 |
| tm-08 | H-AEKSEEEVHNLQKRM-OH | SEQ ID NO: 608 |
| tm-09 | H-EEVHNLQKRMQQLEN-OH | SEQ ID NO: 609 |
| tm-10 | H-LQKRMQQLENDLDQV-OH | SEQ ID NO: 610 |
| tm-11 | H-QQLENDLDQVQESLL-OH | SEQ ID NO: 611 |
| tm-12 | H-DLDQVQESLLKANIQ-OH | SEQ ID NO: 612 |
| tm-13 | H-QESLLKANIQLVEKD-OH | SEQ ID NO: 613 |
| tm-14 | H-KANIQLVEKDKALSN-OH | SEQ ID NO: 614 |
| tm-15 | H-LVEKDKALSNAEGEV-OH | SEQ ID NO: 615 |
| tm-16 | H-KALSNAEGEVAALNR-OH | SEQ ID NO: 616 |
| tm-17 | H-AEGEVAALNRRIQLL-OH | SEQ ID NO: 617 |
| tm-18 | H-AALNRRIQLLEEDLE-OH | SEQ ID NO: 618 |
| tm-19 | H-RIQLLEEDLERSEER-OH | SEQ ID NO: 619 |
| tm-20 | H-EEDLERSEERLNTAT-OH | SEQ ID NO: 620 |
| tm-21 | H-RSEERLNTATTKLAE-OH | SEQ ID NO: 621 |
| tm-22 | H-LNTATTKLAEASQAA-OH | SEQ ID NO: 622 |
| tm-23 | H-TKLAEASQAADESER-OH | SEQ ID NO: 623 |
| tm-24 | H-ASQAADESERMRKVL-OH | SEQ ID NO: 624 |
| tm-25 | H-DESERMRKVLENRSL-OH | SEQ ID NO: 625 |
| tm-26 | H-MRKVLENRSLSDEER-OH | SEQ ID NO: 626 |
| tm-27 | H-ENRSLSDEERMDALE-OH | SEQ ID NO: 627 |
| tm-28 | H-SDEERMDALENQLKE-OH | SEQ ID NO: 628 |
| tm-29 | H-MDALENQLKEARFLA-OH | SEQ ID NO: 629 |
| tm-30 | H-NQLKEARFLAEEADR-OH | SEQ ID NO: 630 |
| tm-31 | H-ARFLAEEADRKYDEV-OH | SEQ ID NO: 631 |
| tm-32 | H-EEADRKYDEVARKLA-OH | SEQ ID NO: 632 |
| tm-33 | H-KYDEVARKLAMVEAD-OH | SEQ ID NO: 633 |
| tm-34 | H-ARKLAMVEADLERAE-OH | SEQ ID NO: 634 |
| tm-35 | H-MVEADLERAEERAET-OH | SEQ ID NO: 635 |
| tm-36 | H-LERAEERAETGESKI-OH | SEQ ID NO: 636 |
| tm-37 | H-ERAETGESKIVELEE-OH | SEQ ID NO: 637 |
| tm-38 | H-GESKIVELEEELRVV-OH | SEQ ID NO: 638 |
| tm-39 | H-VELEEELRVVGNNLK-OH | SEQ ID NO: 639 |
| tm-40 | H-ELRVVGNNLKSLEVS-OH | SEQ ID NO: 640 |
| tm-41 | H-GNNLKSLEVSEEKAN-OH | SEQ ID NO: 641 |
| tm-42 | H-SLEVSEEKANQREEA-OH | SEQ ID NO: 642 |
| tm-43 | H-EEKANQREEAYKEQI-OH | SEQ ID NO: 643 |
| tm-44 | H-QREEAYKEQIKTLTN-OH | SEQ ID NO: 644 |
| tm-45 | H-YKEQIKTLTNKLKAA-OH | SEQ ID NO: 645 |
| tm-46 | H-KTLTNKLKAAEARAE-OH | SEQ ID NO: 646 |
| tm-47 | H-KLKAAEARAEFAERS-OH | SEQ ID NO: 647 |
| tm-48 | H-EARAEFAERSVQKLQ-OH | SEQ ID NO: 648 |
| tm-49 | H-FAERSVQKLQKEVDR-OH | SEQ ID NO: 649 |
| tm-50 | H-VQKLQKEVDRLEDEL-OH | SEQ ID NO: 650 |
| tm-51 | H-KEVDRLEDELVNEKE-OH | SEQ ID NO: 651 |
| tm-52 | H-LEDELVNEKEKYKSI-OH | SEQ ID NO: 652 |
| tm-53 | H-VNEKEKYKSITDELD-OH | SEQ ID NO: 653 |
| tm-54 | H-KYKSITDELDQTFSE-OH | SEQ ID NO: 654 |
| tm-55 | H-TDELDQTFSELSGY-OH | SEQ ID NO: 655 |
| tpc-01 | H-MDSLDEEQIETLRKA-OH | SEQ ID NO: 656 |
| tpc-02 | H-EEQIETLRKAFDSFD-OH | SEQ ID NO: 657 |
| tpc-03 | H-TLRKAFDSFDTEKTG-OH | SEQ ID NO: 658 |
| tpc-04 | H-FDSFDTEKTGSITAE-OH | SEQ ID NO: 659 |
| tpc-05 | H-TEKTGSITAETIATI-OH | SEQ ID NO: 660 |
| tpc-06 | H-SITAETIATIMRMMG-OH | SEQ ID NO: 661 |
| tpc-07 | H-TIATIMRMMGVKISE-OH | SEQ ID NO: 662 |
| tpc-08 | H-MRMMGVKISEKNLQE-OH | SEQ ID NO: 663 |
| tpc-09 | H-VKISEKNLQEAIAET-OH | SEQ ID NO: 664 |
| tpc-10 | H-KNLQEAIAETDEDGS-OH | SEQ ID NO: 665 |
| tpc-11 | H-AIAETDEDGSGLLEF-OH | SEQ ID NO: 666 |
| tpc-12 | H-DEDGSGLLEFEEFVE-OH | SEQ ID NO: 667 |
| tpc-13 | H-GLLEFEEFVELSAKF-OH | SEQ ID NO: 668 |
| tpc-14 | H-EEFVELSAKFLIEED-OH | SEQ ID NO: 669 |
| tpc-15 | H-LSAKFLIEEDEEALK-OH | SEQ ID NO: 670 |
| tpc-16 | H-LIEEDEEALKAELRE-OH | SEQ ID NO: 671 |
| tpc-17 | H-EEALKAELREAFRIY-OH | SEQ ID NO: 672 |
| tpc-18 | H-AELREAFRIYDKEGN-OH | SEQ ID NO: 673 |
| tpc-19 | H-AFRIYDKEGNGFITT-OH | SEQ ID NO: 674 |
| tpc-20 | H-DKEGNGFITTDVLKE-OH | SEQ ID NO: 675 |
| tpc-21 | H-GFITTDVLKEILAEL-OH | SEQ ID NO: 676 |
| tpc-22 | H-DVLKEILAELDPRLT-OH | SEQ ID NO: 677 |
| tpc-23 | H-ILAELDPRLTPADLE-OH | SEQ ID NO: 678 |
| tpc-24 | H-DPRLTPADLENIIEE-OH | SEQ ID NO: 679 |
| tpc-25 | H-PADLENIIEEVDEDG-OH | SEQ ID NO: 680 |
| tpc-26 | H-NIIEEVDEDGSGTLD-OH | SEQ ID NO: 681 |
| tpc-27 | H-VDEDGSGTLDFDEFM-OH | SEQ ID NO: 682 |
| tpc-28 | H-SGTLDFDEFMEMMNG-OH | SEQ ID NO: 683 |

Accordingly, the invention encompasses a method for diagnosing a food allergy in a subject comprising:
a) providing a plurality of peptides derived from one or more allergenic proteins found in the food, each peptide conjugated to a separately identifiable solid support;
b) contacting each solid support with serum obtained from the subject under conditions sufficient to permit binding of IgE in the serum to the peptide on each solid support to form a peptide-IgE complex;
c) binding an IgE-specific labeling reagent to the peptide-IgE complex; and
d) analyzing binding of the labeling reagent to each peptide-IgE complex to identify peptides recognized by the IgE in the serum of the subject;
   wherein recognition of at least one peptide by IgE in the serum of the subject indicates that the subject is allergic to the food.

In another aspect the invention provides a method for detecting development of clinical tolerance to a food in a subject initially allergic to the food comprising:
a) providing an initial profile of the subject's serum IgE reactivity to a plurality of peptides derived from one or more allergenic proteins found in the food, wherein the initial profile defines an initial number of peptides recognized by IgE in the serum of the subject or an initial concentration of IgE in the serum of the subject that recognizes each peptide;
b) providing the plurality of peptides each conjugated to a separately identifiable solid support;
c) contacting each solid support with serum obtained from the subject at a time-point subsequent to the initial profile under conditions sufficient to permit binding of IgE in the serum to the peptide on each solid support to form a peptide-IgE complex;
d) binding an IgE-specific labeling reagent to the peptide-IgE complex; and
e) analyzing binding of the labeling reagent to each peptide-IgE complex to identify a subsequent number of peptides recognized by IgE in the serum of the subject or a subsequent concentration of IgE in the serum of the subject that recognizes each peptide;
   wherein development of clinical tolerance to the food is indicated when the subsequent number of peptides recognized by IgE in the serum of the subject is less than the initial number of peptides recognized by IgE in the serum of the subject, or when the subsequent concentration of IgE in the serum of the subject that recognizes at least one peptide is less than the initial concentration of IgE in the serum of the subject that recognizes the at least one peptide.

In a further aspect, the invention provides a method for detecting an increase in intensity of allergy to cow's milk in a subject over time, the method comprising:
a) providing an initial profile of the subject's serum IgE reactivity to a plurality of peptides derived from one or more allergenic proteins found in the food, wherein the initial profile defines an initial number of peptides recognized by IgE in the serum of the subject or an initial concentration of IgE in the serum of the subject that recognizes each peptide;
b) providing the plurality of peptides each conjugated to a separately identifiable solid support
c) contacting each solid support with serum obtained from the subject at a time-point subsequent to the initial profile under conditions sufficient to permit binding of IgE in the serum to the peptide on each solid support to form a peptide-IgE complex;
d) binding an IgE-specific labeling reagent to the peptide-IgE complex; and
e) analyzing the binding of the labeling reagent to each peptide-IgE complex to identify a subsequent number of peptides recognized by IgE in the serum of the subject or a subsequent concentration of IgE in the serum of the subject that recognizes each peptide;
   wherein an increase in the subsequent number of peptides recognized by IgE in the serum of the subject compared to the initial number of peptides recognized by IgE in the serum of the subject, or an increase in the subsequent concentration of IgE in the serum of the subject that recognizes at least one peptide compared to the initial concentration of IgE in the serum of the subject that recognizes the at least one peptide, indicates increased intensity in the subject of the allergic response to the food.

The reagents and materials used in any of the foregoing methods may be packaged in the form of a kit in which the plurality of allergenic epitope-containing peptides, a labeling reagent comprising an anti-IgE antibody conjugated to a first reporter moiety and, optionally, a second reporter moiety that specifically binds to the labeling reagent are packaged together.

It will also be understood that any of the peptide panels disclosed herein, and subsets thereof, that are useful in the methods of the invention are also an aspect of the invention.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. A method for diagnosing an egg allergy in a subject comprising:
a) providing a plurality of peptides selected derived from one or more allergenic proteins found in the egg, each peptide conjugated to a separately identifiable solid support;
b) contacting each solid support with serum obtained from the subject under conditions sufficient to permit binding of allergy associated immunoglobulin (AAI) in the serum to the peptide on each solid support to form a peptide-IgE complex;
c) binding an AAI-specific labeling reagent to the peptide-AAI complex; and
d) analyzing binding of the labeling reagent to each peptide-AAI complex to identify peptides recognized by the AAI in the serum of the subject;
wherein recognition of at least one peptide by AAI in the serum of the subject indicates that the subject is allergic to eggs.

2. (canceled)

3. The method of claim 1, wherein IgG and/or IgE are detected.

4. The method of claim 3 wherein the IgG is IgG4.

5-7. (canceled)

8. The method of claim 1, wherein the plurality of peptides is selected from among SEQ ID NOs: 278-460.

9. (canceled)

10. A method for detecting development of clinical tolerance to eggs in a subject that is allergic to eggs comprising:
a) providing an initial profile of allergy associated immunoglobulin (AAI) reactivity of the subject's serum to a plurality of peptides derived from one or more allergenic proteins found in eggs, wherein the initial profile defines an initial number of peptides recognized by AAI in the serum of the subject or an initial concentration of AAI in the serum of the subject that recognizes each peptide;
b) providing the plurality of peptides each conjugated to a separately identifiable solid support;
c) contacting each solid support with serum obtained from the subject at a time-point subsequent to the initial profile under conditions sufficient to permit binding of AAI in the serum to the peptide on each solid support to form a peptide-AAI complex;
d) binding an AAI-specific labeling reagent to the peptide-AAI complex; and
e) analyzing binding of the labeling reagent to each peptide-AAI complex to identify a subsequent number of peptides recognized by AAI in the serum of the subject or a subsequent concentration of AAI in the serum of the subject that recognizes each peptide;
wherein development of clinical tolerance to eggs is indicated when the subsequent number of peptides recognized by AAI in the serum of the subject is less than the initial number of peptides recognized by AAI in the serum of the subject, or when the subsequent concentration of AAI in the serum of the subject that recognizes at least one peptide is less than the initial concentration of AAI in the serum of the subject that recognizes the at least one peptide.

11-14. (canceled)

15. The method of claim 10, wherein the plurality of peptides is selected from among SEQ ID NOs: 278-460.

16. (canceled)

17. The method of claim 10, wherein a pattern of quantitative reduction in AAI reactivity with selected peptides is used to predict development of clinical tolerance to the allergenic eggs in the subject.

18. A method for detecting an increase in intensity of allergy to eggs over time in a subject that is allergic to eggs, the method comprising:
a) providing an initial profile of reactivity of allergy associated immunoglobulin (AAI) in the subject's serum to a plurality of peptides derived from one or more allergenic proteins found in eggs, wherein the initial profile defines an initial number of peptides recognized by AAI in the serum of the subject or an initial concentration of AAI in the serum of the subject that recognizes each peptide;
b) providing the plurality of peptides each conjugated to a separately identifiable solid support;
c) contacting each solid support with serum obtained from the subject at a time-point subsequent to the initial profile under conditions sufficient to permit binding of AAI in the serum to the peptide on each solid support to form a peptide-AAI complex;
d) binding an AAI-specific labeling reagent to the peptide-AAI complex; and
e) analyzing the binding of the labeling reagent to each peptide-AAI complex to identify a subsequent number of peptides recognized by AAI in the serum of the subject or a subsequent concentration of AAI in the serum of the subject that recognizes each peptide;
wherein an increase in the subsequent number of peptides recognized by AAI in the serum of the subject compared to the initial number of peptides recognized by AAI in the serum of the subject, or an increase in the subsequent concentration of AAI in the serum of the subject that recognizes at least one peptide compared to the initial concentration of AAI in the serum of the subject that recognizes the at least one peptide, indicates increased intensity of the allergic response to eggs in the subject.

19-22. (canceled)

23. The method of claim 18, wherein the plurality of peptides is selected from among SEQ ID NOs: 278-460.

24. (canceled)

25. The method of claim 18, wherein a pattern of quantitatively increased AAI reactivity with selected peptides is used to predict increasing intensity of allergy to eggs over time.

26-28. (canceled)

29. A set of allergenic epitope-containing peptides for detection of egg allergy comprising a plurality of peptides selected from the group consisting of peptides represented by SEQ ID NOs: 278-460 and/or a plurality of peptides selected from the group consisting of peptides represented by twelve or more contiguous amino acids of SEQ ID NOs: 278-460.

30. (canceled)

31. A kit for detection of egg allergy, detection of an increase or decrease in intensity of egg allergy over time, or detection of development of clinical tolerance to allergenic egg proteins comprising, packaged together and including instructions for use:
a) a plurality of allergenic epitope-containing peptides derived from one or more allergenic proteins found in eggs;
b) a labeling reagent comprising an anti-allergy associated immunoglobulin antibody (anti-AAI) conjugated to a first reporter moiety; and
c) optionally, a second reporter moiety that specifically binds to the labeling reagent.

32. The kit of claim 31, wherein the instructions are for use of the kit to predict a patient's natural tolerance, responsiveness to therapy, or increase in allergic response.

33. The kit of claim 32 which comprises the set of allergenic epitope-containing peptides of claim 29.

34. (canceled)

35. The method of claim 1, which is a multiplex peptide-bead assay for flow cytometric analysis or a lateral flow assay.

36. The method of claim 10, which is a multiplex peptide-bead assay for flow cytometric analysis or a lateral flow assay.

37. The method of claim 18, which is a multiplex peptide-bead assay for flow cytometric analysis or a lateral flow assay.


SEQUENCE LISTING (683 sequences)

SEQ ID NO: 1 - protein, 20 aa, synthetic construct
KHQGLPQEVLNENLLRFFVA

SEQ ID NO: 2 - protein, 20 aa, synthetic construct
VAPFPEVFGKEKVNELSKDI

SEQ ID NO: 3 - protein, 20 aa, synthetic construct
SISSSEEIVPNSVEQKHIQK

SEQ ID NO: 4 - protein, 20 aa, synthetic construct
KHIQKEDVPSERYLGYLEQL

SEQ ID NO: 5 - protein, 20 aa, synthetic construct
SERYLGYLEQLLRLKKYKVP

SEQ ID NO: 6 - protein, 20 aa, synthetic construct
KYKVPQLEIVPNSAEERLHS

SEQ ID NO: 7 - protein, 20 aa, synthetic construct
QQKEPMIGVNQELAYFYPEL

SEQ ID NO: 8 - protein, 20 aa, synthetic construct
LGTQYTDAPSFSDIPNPIGS

SEQ ID NO: 9 - protein, 20 aa, synthetic construct
SDIPNPIGSENSEKTTMPLW

SEQ ID NO: 10 - protein, 20 aa, synthetic construct
QEKNMAINPSKENLCSTFCK

SEQ ID NO: 11 - protein, 20 aa, synthetic construct
STFCKEVVRNANEEEYSIGS

SEQ ID NO: 12 - protein, 20 aa, synthetic construct
KHYQKALNEINQFYQKFPQY

SEQ ID NO: 13 - protein, 20 aa, synthetic construct
QYLYQGPIVLNPWDQVKRNA

SEQ ID NO: 14 - protein, 20 aa, synthetic construct
KISQRYQKFALPQYLKTVYQ

SEQ ID NO: 15 - protein, 20 aa, synthetic construct
QYLKTVYQHQKAMKPWIQPK

SEQ ID NO: 16 - protein, 20 aa, synthetic construct
RELEELNVPGEIVESLSSSE

SEQ ID NO: 17 - protein, 20 aa, synthetic construct
QDKIHPFAQTQSLVYPFPGP

SEQ ID NO: 18 - protein, 20 aa, synthetic construct
FAQTQSLVYPFPGPIPNSLP

SEQ ID NO: 19 - protein, 20 aa, synthetic construct
NIPPLTQTPVVVPPFLQPEV

SEQ ID NO: 20 - protein, 20 aa, synthetic construct
KVKEAMAPKHKEMPFPKYPV

SEQ ID NO: 21 - protein, 20 aa, synthetic construct
SLTLTDVENLHLPLPLLQSW

SEQ ID NO: 22 - protein, 20 aa, synthetic construct
FPPQSVLSLSQSKVLPVPQK

SEQ ID NO: 23 - protein, 20 aa, synthetic construct
PVPQKAVPYPQRDMPIQAFL

SEQ ID NO: 24 - protein, 20 aa, synthetic construct
RVYVEELKPTPEGDLEILLQ

SEQ ID NO: 25 - protein, 20 aa, synthetic construct
DECAQKKIIAEKTKIPAVFK

SEQ ID NO: 26 - protein, 20 aa, synthetic construct
CLVRTPEVDDEALEKFDKAL

SEQ ID NO: 27 - protein, 20 aa, synthetic construct
EVDDEALEKFDKALKALPMH

SEQ ID NO: 28 - protein, 20 aa, synthetic construct
RCEKDERFFSDKIAKYIPIQ

SEQ ID NO: 29 - protein, 20 aa, synthetic construct
KPVALINNQFLPYPYYAKPA

SEQ ID NO: 30 - protein, 20 aa, synthetic construct
MAIPPKKNQDKTEIPTINTI

SEQ ID NO: 31 - protein, 20 aa, synthetic construct
PTSTPTTEAVESTVATLEDS

SEQ ID NO: 32 - protein, 20 aa, synthetic construct
TLEDSPEVIESPPEINTVQV

SEQ ID NO: 33 - protein, 20 aa, synthetic construct
YAKPAAVRSPAQILQWQVLS

SEQ ID NO: 34 - protein, 20 aa, synthetic construct
NREQLSTSEENSKKTVDMES

SEQ ID NO: 35 - protein, 20 aa, synthetic construct
RINKKIEKFQSEEQQQTEDE

SEQ ID NO: 36 - protein, 20 aa, synthetic construct
KVLVLDTDYKKYLLVCMENS

SEQ ID NO: 37 - protein, 20 aa, synthetic construct
NLLRFFVAPFPEVFGKEKVN

SEQ ID NO: 38 - protein, 20 aa, synthetic construct
PNSVEQKHIQKEDVPSERYL

SEQ ID NO: 39 - protein, 20 aa, synthetic construct
QKEDVPSERYLGYLEQLLRL

SEQ ID NO: 40 - protein, 20 aa, synthetic construct
LEIVPNSAEERLHSMKEGIH

SEQ ID NO: 41 - protein, 20 aa, synthetic construct
ERLHSMKEGIHAQQKEPMIG

SEQ ID NO: 42 - protein, 20 aa, synthetic construct
IHAQQKEPMIGVNQELAYFY

SEQ ID NO: 43 - protein, 20 aa, synthetic construct
IGVNQELAYFYPELFRQFYQ

SEQ ID NO: 44 - protein, 20 aa, synthetic construct
PSGAWYYVPLGTQYTDAPSF

SEQ ID NO: 45 - protein, 20 aa, synthetic construct
AWYYVPLGTQYTDAPSFSDI

SEQ ID NO: 46 - protein, 20 aa, synthetic construct
SIISQETYKQEKNMAINPSK

SEQ ID NO: 47 - protein, 20 aa, synthetic construct
INPSKENLCSTFCKEVVRNA

SEQ ID NO: 48 - protein, 20 aa, synthetic construct
SAEVATEEVKITVDDKHYQK

SEQ ID NO: 49 - protein, 20 aa, synthetic construct
TSEENSKKTVDMESTEVFTK

SEQ ID NO: 50 - protein, 20 aa, synthetic construct
TKLTEEEKNRLNFLKKISQR

SEQ ID NO: 51 - protein, 20 aa, synthetic construct
YQHQKAMKPWIQPKTKVIPY

SEQ ID NO: 52 - protein, 20 aa, synthetic construct
PFPGPIPNSLPQNIPPLTQT

SEQ ID NO: 53 - protein, 20 aa, synthetic construct
QTPVVVPPFLQPEVMGVSKV

SEQ ID NO: 54 - protein, 20 aa, synthetic construct
VENLHLPLPLLQSWMHQPHQ

SEQ ID NO: 55 - protein, 20 aa, synthetic construct
SWMHQPHQPLPPTVMFPPQS

SEQ ID NO: 56 - protein, 20 aa, synthetic construct
SQSKVLPVPQKAVPYPQRDM

SEQ ID NO: 57 - protein, 20 aa, synthetic construct
GDLEILLQKWENDECAQKKI

SEQ ID NO: 58 - protein, 20 aa, synthetic construct
DEALEKFDKALKALPMHIRL

SEQ ID NO: 59 - protein, 20 aa, synthetic construct
RFFSDKIAKYIPIQYVLSRY

SEQ ID NO: 60 - protein, 20 aa, synthetic construct
KPVALINNQFLPYPYYAKPA

SEQ ID NO: 61 - protein, 20 aa, synthetic construct
YAKPAAVRSPAQILQWQVLS

SEQ ID NO: 62 - protein, 20 aa, synthetic construct
PAQILQWQVLSNTVPAKSCQ

SEQ ID NO: 63 - protein, 20 aa, synthetic construct
CQAQPTTMARHPHPHLSFMA

SEQ ID NO: 64 - protein, 20 aa, synthetic construct
RHPHPHLSFMAIPPKKNQDK

SEQ ID NO: 65 - protein, 20 aa, synthetic construct
TINTIASGEPTSTPTTEAVE

SEQ ID NO: 66 - protein, 20 aa, synthetic construct
DSPEVIESPPEINTVQVTST

SEQ ID NO: 67 - protein, 20 aa, synthetic construct
PEVIESPPEINTVQVTSTAV

SEQ ID NO: 68 - protein, 20 aa, synthetic construct
QSLVCQCLVRTPEVDDEALE

SEQ ID NO: 69 - protein, 15 aa, synthetic construct
VLASVSATHAKSSPY

SEQ ID NO: 70 - protein, 15 aa, synthetic construct
SVSATHAKSSPYQKK

SEQ ID NO: 71 - protein, 15 aa, synthetic construct
ATHAKSSPYQKKTEN

SEQ ID NO: 72 - protein, 15 aa, synthetic construct
TENPCAQRCLQSCQQ

SEQ ID NO: 73 - protein, 15 aa, synthetic construct
PCAQRCLQSCQQEPD

SEQ ID NO: 74 - protein, 15 aa, synthetic construct
LQSCQQEPDDLKQKA

SEQ ID NO: 75 - protein, 15 aa, synthetic construct
CQQEPDDLKQKACES

SEQ ID NO: 76 - protein, 15 aa, synthetic construct
EPDDLKQKACESRCT

SEQ ID NO: 77 - protein, 15 aa, synthetic construct
QKACESRCTKLEYDP

SEQ ID NO: 78 - protein, 15 aa, synthetic construct
CESRCTKLEYDPRCV

SEQ ID NO: 79 - protein, 15 aa, synthetic construct
RCTKLEYDPRCVYDP

SEQ ID NO: 80 - protein, 15 aa, synthetic construct
KLEYDPRCVYDPRGH

SEQ ID NO: 81 - protein, 15 aa, synthetic construct
YDPRCVYDPRGHTGT

SEQ ID NO: 82 - protein, 15 aa, synthetic construct
YDPRGHTGTTNQRSP

SEQ ID NO: 83 - protein, 15 aa, synthetic construct
RGHTGTTNQRSPPGE

SEQ ID NO: 84 - protein, 15 aa, synthetic construct
TNQRSPPGERTRGRQ

SEQ ID NO: 85 - protein, 15 aa, synthetic construct
RSPPGERTRGRQPGD

SEQ ID NO: 86 - protein, 15 aa, synthetic construct
PGERTRGRQPGDYDD

SEQ ID NO: 87 - protein, 15 aa, synthetic construct
RTRGRQPGDYDDDRR

SEQ ID NO: 88 - protein, 15 aa, synthetic construct
GRQPGDYDDDRRQPR

SEQ ID NO: 89 - protein, 15 aa, synthetic construct
PGDYDDDRRQPRREE

SEQ ID NO: 90 - protein, 15 aa, synthetic construct
YDDDRRQPRREEGGR

SEQ ID NO: 91 - protein, 15 aa, synthetic construct
DRRQPRREEGGRWGP

SEQ ID NO: 92 - protein, 15 aa, synthetic construct
QPRREEGGRWGPAGP

SEQ ID NO: 93 - protein, 15 aa, synthetic construct
REEGGRWGPAGPRER

SEQ ID NO: 94 - protein, 15 aa, synthetic construct
GGRWGPAGPRERERE

SEQ ID NO: 95 - protein, 15 aa, synthetic construct
WGPAGPREREREEDW

SEQ ID NO: 96 - protein, 15 aa, synthetic construct
AGPREREREEDWRQP

SEQ ID NO: 97 - protein, 15 aa, synthetic construct
REREREEDWRQPRED

SEQ ID NO: 98 - protein, 15 aa, synthetic construct
EREEDWRQPREDWRR

SEQ ID NO: 99 - protein, 15 aa, synthetic construct
EDWRQPREDWRRPSH

SEQ ID NO: 100 - protein, 15 aa, synthetic construct
RQPREDWRRPSHQQP

SEQ ID NO: 101 - protein, 15 aa, synthetic construct
REDWRRPSHQQPRKI

SEQ ID NO: 102 - protein, 15 aa, synthetic construct
WRRPSHQQPRKIRPE

SEQ ID NO: 103 - protein, 15 aa, synthetic construct
PSHQQPRKIRPEGRE

SEQ ID NO: 104 - protein, 15 aa, synthetic construct
QQPRKIRPEGREGEQ

SEQ ID NO: 105 - protein, 15 aa, synthetic construct
RKIRPEGREGEQEWG

SEQ ID NO: 106 - protein, 15 aa, synthetic construct
RPEGREGEQEWGTPG

SEQ ID NO: 107 - protein, 15 aa, synthetic construct
GREGEQEWGTPGSHV

SEQ ID NO: 108 - protein, 15 aa, synthetic construct
GEQEWGTPGSHVREE

SEQ ID NO: 109 - protein, 15 aa, synthetic construct
EWGTPGSHVREETSR

SEQ ID NO: 110 - protein, 15 aa, synthetic construct
REETSRNNPFYFPSR

SEQ ID NO: 111 - protein, 15 aa, synthetic construct
TSRNNPFYFPSRRFS

SEQ ID NO: 112 - protein, 15 aa, synthetic construct
NNPFYFPSRRFSTRY

SEQ ID NO: 113 - protein, 15 aa, synthetic construct
RIPSGFISYILNRHD

SEQ ID NO: 114 - protein, 15 aa, synthetic construct
SGFISYILNRHDNQN

SEQ ID NO: 115 - protein, 15 aa, synthetic construct
NQNLRVAKISMPVNT

SEQ ID NO: 116 - protein, 15 aa, synthetic construct
LRVAKISMPVNTPGQ

SEQ ID NO: 117 - protein, 15 aa, synthetic construct
AKISMPVNTPGQFED

SEQ ID NO: 118 - protein, 15 aa, synthetic construct
SMPVNTPGQFEDFFP

SEQ ID NO: 119 - protein, 15 aa, synthetic construct
VNTPGQFEDFFPASS

SEQ ID NO: 120 - protein, 15 aa, synthetic construct
PGQFEDFFPASSRDQ

SEQ ID NO: 121 - protein, 15 aa, synthetic construct
FEDFFPASSRDQSSY

SEQ ID NO: 122 - protein, 15 aa, synthetic construct
ASSRDQSSYLQGFSR

SEQ ID NO: 123 - protein, 15 aa, synthetic construct
RDQSSYLQGFSRNTL

SEQ ID NO: 124 - protein, 15 aa, synthetic construct
SSYLQGFSRNTLEAA

SEQ ID NO: 125 - protein, 15 aa, synthetic construct
LQGFSRNTLEAAFNA

SEQ ID NO: 126 - protein, 15 aa, synthetic construct
RVLLEENAGGEQEER

SEQ ID NO: 127 - protein, 15 aa, synthetic construct
LEENAGGEQEERGQR

SEQ ID NO: 128 - protein, 15 aa, synthetic construct
NAGGEQEERGQRRWS

SEQ ID NO: 129 - protein, 15 aa, synthetic construct
GEQEERGQRRWSTRS

SEQ ID NO: 130 - protein, 15 aa, synthetic construct
EERGQRRWSTRSSEN

SEQ ID NO: 131 - protein, 15 aa, synthetic construct
KKGSEEEGDITNPIN

SEQ ID NO: 132 - protein, 15 aa, synthetic construct
SEEEGDITNPINLRE

SEQ ID NO: 133 - protein, 15 aa, synthetic construct
EGDITNPINLREGEP

SEQ ID NO: 134 - protein, 15 aa, synthetic construct
ITNPINLREGEPDLS

SEQ ID NO: 135 - protein, 15 aa, synthetic construct
PINLREGEPDLSNNF

SEQ ID NO: 136 - protein, 15 aa, synthetic construct
LREGEPDLSNNFGKL

SEQ ID NO: 137 - protein, 15 aa, synthetic construct
GEPDLSNNFGKLFEV

SEQ ID NO: 138 - protein, 15 aa, synthetic construct
DLSNNFGKLFEVKPD

SEQ ID NO: 139 - protein, 15 aa, synthetic construct
NNFGKLFEVKPDKKN

SEQ ID NO: 140 - protein, 15 aa, synthetic construct
GKLFEVKPDKKNPQL

SEQ ID NO: 141 - protein, 15 aa, synthetic construct
KKNPQLQDLDMMLTC

SEQ ID NO: 142 - protein, 15 aa, synthetic construct
PQLQDLDMMLTCVEI

SEQ ID NO: 143 - protein, 15 aa, synthetic construct
QDLDMMLTCVEIKEG

SEQ ID NO: 144 - protein, 15 aa, synthetic construct
DMMLTCVEIKEGALM

SEQ ID NO: 145 - protein, 15 aa, synthetic construct
LTCVEIKEGALMLPH

SEQ ID NO: 146 - protein, 15 aa, synthetic construct
VEIKEGALMLPHFNS

SEQ ID NO: 147 - protein, 15 aa, synthetic construct
KEGALMLPHFNSKAM

SEQ ID NO: 148 - protein, 15 aa, synthetic construct
SNREVRRYTARLKEG

SEQ ID NO: 149 - protein, 15 aa, synthetic construct
EVRRYTARLKEGDVF

SEQ ID NO: 150 - protein, 15 aa, synthetic construct
RYTARLKEGDVFIMP

SEQ ID NO: 151 - protein, 15 aa, synthetic construct
ARLKEGDVFIMPAAH

SEQ ID NO: 152 - protein, 15 aa, synthetic construct
KEGDVFIMPAAHPVA

SEQ ID NO: 153 - protein, 15 aa, synthetic construct
DVFIMPAAHPVAINA

SEQ ID NO: 154 - protein, 15 aa, synthetic construct
PVAINASSELHLLGF

SEQ ID NO: 155 - protein, 15 aa, synthetic construct
INASSELHLLGFGIN

SEQ ID NO: 156 - protein, 15 aa, synthetic construct
SSELHLLGFGINAEN

SEQ ID NO: 157 - protein, 15 aa, synthetic construct
LHLLGFGINAENNHR

SEQ ID NO: 158 - protein, 15 aa, synthetic construct
LGFGINAENNHRIFL

SEQ ID NO: 159 - protein, 15 aa, synthetic construct
GINAENNHRIFLAGD

SEQ ID NO: 160 - protein, 15 aa, synthetic construct
AENNHRIFLAGDKDN

SEQ ID NO: 161 - protein, 15 aa, synthetic construct
NHRIFLAGDKDNVID

SEQ ID NO: 162 - protein, 15 aa, synthetic construct
IFLAGDKDNVIDQIE

SEQ ID NO: 163 - protein, 15 aa, synthetic construct
AGDKDNVIDQIEKQA

SEQ ID NO: 164 - protein, 15 aa, synthetic construct
KDNVIDQIEKQAKDL

SEQ ID NO: 165 - protein, 15 aa, synthetic construct
VIDQIEKQAKDLAFP

SEQ ID NO: 166 - protein, 15 aa, synthetic construct
QIEKQAKDLAFPGSG

SEQ ID NO: 167 - protein, 15 aa, synthetic construct
KQAKDLAFPGSGEQV

SEQ ID NO: 168 - protein, 15 aa, synthetic construct
KDLAFPGSGEQVEKL

SEQ ID NO: 169 - protein, 15 aa, synthetic construct
AFPGSGEQVEKLIKN

SEQ ID NO: 170 - protein, 15 aa, synthetic construct
GSGEQVEKLIKNQKE

SEQ ID NO: 171 - protein, 15 aa, synthetic construct
EQVEKLIKNQKESHF

SEQ ID NO: 172 - protein, 15 aa, synthetic construct
EKLIKNQKESHFVSA

SEQ ID NO: 173 - protein, 15 aa, synthetic construct
IKNQKESHFVSARPQ

SEQ ID NO: 174 - protein, 15 aa, synthetic construct
QKESHFVSARPQSQS

SEQ ID NO: 175 - protein, 15 aa, synthetic construct
SHFVSARPQSQSQSP

SEQ ID NO: 176 - protein, 15 aa, synthetic construct
VSARPQSQSQSPSSP

SEQ ID NO: 177 - protein, 15 aa, synthetic construct
RPQSQSQSPSSPEKE

SEQ ID NO: 178 - protein, 15 aa, synthetic construct
SQSQSPSSPEKESPE

SEQ ID NO: 179 - protein, 15 aa, synthetic construct
QSPSSPEKESPEKED

SEQ ID NO: 180 - protein, 15 aa, synthetic construct
SSPEKESPEKEDQEE

SEQ ID NO: 181 - protein, 15 aa, synthetic construct
EKESPEKEDQEEENQ

SEQ ID NO: 182 - protein, 15 aa, synthetic construct
SPEKEDQEEENQGGK

SEQ ID NO: 183 - protein, 15 aa, synthetic construct
KEDQEEENQGGKGPL

SEQ ID NO: 184 - protein, 15 aa, synthetic construct
QEEENQGGKGPLLSI

SEQ ID NO: 185 - protein, 15 aa, synthetic construct
AAHASARQQWELQGD

SEQ ID NO: 186 - protein, 15 aa, synthetic construct
ASARQQWELQGDRRC

SEQ ID NO: 187 - protein, 15 aa, synthetic construct
RQQWELQGDRRCQSQ

SEQ ID NO: 188 - protein, 15 aa, synthetic construct
WELQGDRRCQSQLER

SEQ ID NO: 189 - protein, 15 aa, synthetic construct
QGDRRCQSQLERANL

SEQ ID NO: 190 - protein, 15 aa, synthetic construct
RRCQSQLERANLRPC

SEQ ID NO: 191 - protein, 15 aa, synthetic construct
QSQLERANLRPCEQH

SEQ ID NO: 192 - protein, 15 aa, synthetic construct
LERANLRPCEQHLMQ

SEQ ID NO: 193 - protein, 15 aa, synthetic construct
ANLRPCEQHLMQKIQ

SEQ ID NO: 194 - protein, 15 aa, synthetic construct
RPCEQHLMQKIQRDE

SEQ ID NO: 195 - protein, 15 aa, synthetic construct
EQHLMQKIQRDEDSY

SEQ ID NO: 196 - protein, 15 aa, synthetic construct
LMQKIQRDEDSYERD

SEQ ID NO: 197 - protein, 15 aa, synthetic construct
KIQRDEDSYERDPYS

SEQ ID NO: 198 - protein, 15 aa, synthetic construct
RDEDSYERDPYSPSQ

SEQ ID NO: 199 - protein, 15 aa, synthetic construct
DSYERDPYSPSQDPY

SEQ ID NO: 200 - protein, 15 aa, synthetic construct
ERDPYSPSQDPYSPS

SEQ ID NO: 201 - protein, 15 aa, synthetic construct
PYSPSQDPYSPSPYD

SEQ ID NO: 202 - protein, 15 aa, synthetic construct
PSQDPYSPSPYDRRG

SEQ ID NO: 203 - protein, 15 aa, synthetic construct
DPYSPSPYDRRGAGS

SEQ ID NO: 204 - protein, 15 aa, synthetic construct
SPSPYDRRGAGSSQH

SEQ ID NO: 205 - protein, 15 aa, synthetic construct
PYDRRGAGSSQHQER

SEQ ID NO: 206 - protein, 15 aa, synthetic construct
QERCCNELNEFENNQ

SEQ ID NO: 207 - protein, 15 aa, synthetic construct
CCNELNEFENNQRCM

SEQ ID NO: 208 - protein, 15 aa, synthetic construct
ELNEFENNQRCMCEA

SEQ ID NO: 209 - protein, 15 aa, synthetic construct
EFENNQRCMCEALQQ

SEQ ID NO: 210 - protein, 15 aa, synthetic construct
RCMCEALQQIMENQS

SEQ ID NO: 211 - protein, 15 aa, synthetic construct
CEALQQIMENQSDRL

SEQ ID NO: 212 - protein, 15 aa, synthetic construct
LQQIMENQSDRLQGR

SEQ ID NO: 213 - protein, 15 aa, synthetic construct
IMENQSDRLQGRQQE

SEQ ID NO: 214 - protein, 15 aa, synthetic construct
NQSDRLQGRQQEQQF

SEQ ID NO: 215 - protein, 15 aa, synthetic construct
DRLQGRQQEQQFKRE

SEQ ID NO: 216 - protein, 15 aa, synthetic construct
QGRQQEQQFKRELRN

SEQ ID NO: 217 - protein, 15 aa, synthetic construct
QQEQQFKRELRNLPQ

SEQ ID NO: 218 - protein, 15 aa, synthetic construct
QQFKRELRNLPQQCG

SEQ ID NO: 219 - protein, 15 aa, synthetic construct
KRELRNLPQQCGLRA

SEQ ID NO: 220 - protein, 15 aa, synthetic construct
LPQQCGLRAPQRCDL

SEQ ID NO: 221 - protein, 15 aa, synthetic construct
QCGLRAPQRCDLDVE

SEQ ID NO: 222 - protein, 15 aa, synthetic construct
LRAPQRCDLDVESGG

SEQ ID NO: 223 - protein, 15 aa, synthetic construct
RIESEGGYIETWNPN

SEQ ID NO: 224 - protein, 15 aa, synthetic construct
NQEFECAGVALSRLV

SEQ ID NO: 225 - protein, 15 aa, synthetic construct
AGVALSRLVLRRNAL

SEQ ID NO: 226 - protein, 15 aa, synthetic construct
ALSRLVLRRNALRRP

SEQ ID NO: 227 - protein, 15 aa, synthetic construct
RLVLRRNALRRPFYS

SEQ ID NO: 228 - protein, 15 aa, synthetic construct
LRRNALRRPFYSNAP

SEQ ID NO: 229 - protein, 15 aa, synthetic construct
NALRRPFYSNAPQEI

SEQ ID NO: 230 - protein, 15 aa, synthetic construct
HYEEPHTQGRRSQSQ

SEQ ID NO: 231 - protein, 15 aa, synthetic construct
EPHTQGRRSQSQRPP

SEQ ID NO: 232 - protein, 15 aa, synthetic construct
TQGRRSQSQRPPRRL

SEQ ID NO: 233 - protein, 15 aa, synthetic construct
RRSQSQRPPRRLQGE

SEQ ID NO: 234 - protein, 15 aa, synthetic construct
RRLQGEDQSQQQRDS

SEQ ID NO: 235 - protein, 15 aa, synthetic construct
QGEDQSQQQRDSHQK

SEQ ID NO: 236 - protein, 15 aa, synthetic construct
NTEQEFLRYQQQSRQ

SEQ ID NO: 237 - protein, 15 aa, synthetic construct
QEFLRYQQQSRQSRR

SEQ ID NO: 238 - protein, 15 aa, synthetic construct
PYSPQSQPRQEEREF

SEQ ID NO: 239 - protein, 15 aa, synthetic construct
PQSQPRQEEREFSPR

SEQ ID NO: 240 - protein, 15 aa, synthetic construct
QPRQEEREFSPRGQH

SEQ ID NO: 241 - protein, 15 aa, synthetic construct
QEEREFSPRGQHSRR

SEQ ID NO: 242 - protein, 15 aa, synthetic construct
SPRGQHSRRERAGQE

SEQ ID NO: 243 - protein, 15 aa, synthetic construct
GQHSRRERAGQEEEN

SEQ ID NO: 244 - protein, 15 aa, synthetic construct
SRRERAGQEEENEGG

SEQ ID NO: 245 - protein, 15 aa, synthetic construct
GQEEENEGGNIFSGF

SEQ ID NO: 246 - protein, 15 aa, synthetic construct
EENEGGNIFSGFTPE

SEQ ID NO: 247 - protein, 15 aa, synthetic construct
EGGNIFSGFTPEFLE

SEQ ID NO: 248 - protein, 15 aa, synthetic construct
NIFSGFTPEFLEQAF

SEQ ID NO: 249 - protein, 15 aa, synthetic construct
SGFTPEFLEQAFQVD

SEQ ID NO: 250 - protein, 15 aa, synthetic construct
TPEFLEQAFQVDDRQ

SEQ ID NO: 251 - protein, 15 aa, synthetic construct
ESEEEGAIVTVRGGL

SEQ ID NO: 252 - protein, 15 aa, synthetic construct
EEGAIVTVRGGLRIL

SEQ ID NO: 253 - protein, 15 aa, synthetic construct
AIVTVRGGLRILSPD

SEQ ID NO: 254 - protein, 15 aa, synthetic construct
TVRGGLRILSPDRKR

SEQ ID NO: 255 - protein, 15 aa, synthetic construct
GGLRILSPDRKRRAD

SEQ ID NO: 256 - protein, 15 aa, synthetic construct
RILSPDRKRRADEEE

SEQ ID NO: 257 - protein, 15 aa, synthetic construct
RKRRADEEEEYDEDE

SEQ ID NO: 258 - protein, 15 aa, synthetic construct
RADEEEEYDEDEYEY

SEQ ID NO: 259 - protein, 15 aa, synthetic construct
EEEEYDEDEYEYDEE

SEQ ID NO: 260 - protein, 15 aa, synthetic construct
EYDEDEYEYDEEDRR

SEQ ID NO: 261 - protein, 15 aa, synthetic construct
EDEYEYDEEDRRRGR

SEQ ID NO: 262 - protein, 15 aa, synthetic construct
YEYDEEDRRRGRGSR

SEQ ID NO: 263 - protein, 15 aa, synthetic construct
DEEDRRRGRGSRGRG

SEQ ID NO: 264 - protein, 15 aa, synthetic construct
DRRRGRGSRGRGNGI

SEQ ID NO: 265 - protein, 15 aa, synthetic construct
RGRGSRGRGNGIEET

SEQ ID NO: 266 - protein, 15 aa, synthetic construct
GSRGRGNGIEETICT

SEQ ID NO: 267 - protein, 15 aa, synthetic construct
GRGNGIEETICTASA

SEQ ID NO: 268 - protein, 15 aa, synthetic construct
NGIEETICTASAKKN

SEQ ID NO: 269 - protein, 15 aa, synthetic construct
IANLAGENSVIDNLP

SEQ ID NO: 270 - protein, 15 aa, synthetic construct
LAGENSVIDNLPEEV

SEQ ID NO: 271 - protein, 15 aa, synthetic construct
ENSVIDNLPEEVVAN

SEQ ID NO: 272 - protein, 15 aa, synthetic construct
VIDNLPEEVVANSYG

SEQ ID NO: 273 - protein, 15 aa, synthetic construct
EQARQLKNNNPFKFF

SEQ ID NO: 274 - protein, 15 aa, synthetic construct
RQLKNNNPFKFFVPP

SEQ ID NO: 275 - protein, 15 aa, synthetic construct
KNNNPFKFFVPPSQQ

SEQ ID NO: 276 - protein, 15 aa, synthetic construct
NPFKFFVPPSQQSPR

SEQ ID NO: 277 - protein, 15 aa, synthetic construct
KFFVPPSQQSPRAVA

SEQ ID NO: 278 - protein, 15 aa, synthetic construct
MGSIGAASMEFCFDV

SEQ ID NO: 279 - protein, 15 aa, synthetic construct
IGAASMEFCFDVFKE

SEQ ID NO: 280 - protein, 15 aa, synthetic construct
ASMEFCFDVFKELKV

SEQ ID NO: 281 - protein, 15 aa, synthetic construct
EFCFDVFKELKVHHA

SEQ ID NO: 282 - protein, 15 aa, synthetic construct
FDVFKELKVHHANEN

SEQ ID NO: 283 - protein, 15 aa, synthetic construct
FKELKVHHANENIFY

SEQ ID NO: 284 - protein, 15 aa, synthetic construct
LKVHHANENIFYCPI

SEQ ID NO: 285 - protein, 15 aa, synthetic construct
HHANENIFYCPIAIM

SEQ ID NO: 286 - protein, 15 aa, synthetic construct
NENIFYCPIAIMSAL

SEQ ID NO: 287 - protein, 15 aa, synthetic construct
IFYCPIAIMSALAMV

SEQ ID NO: 288 - protein, 15 aa, synthetic construct
CPIAIMSALAMVYLG

SEQ ID NO: 289 - protein, 15 aa, synthetic construct
AIMSALAMVYLGAKD

SEQ ID NO: 290 - protein, 15 aa, synthetic construct
SALAMVYLGAKDSTR

SEQ ID NO: 291 - protein, 15 aa, synthetic construct
AMVYLGAKDSTRTQI

SEQ ID NO: 292 - protein, 15 aa, synthetic construct
YLGAKDSTRTQINKV

SEQ ID NO: 293 - protein, 15 aa, synthetic construct
AKDSTRTQINKVVRF

SEQ ID NO: 294 - protein, 15 aa, synthetic construct
STRTQINKVVRFDKL

SEQ ID NO: 295 - protein, 15 aa, synthetic construct
TQINKVVRFDKLPGF

SEQ ID NO: 296 - protein, 15 aa, synthetic construct
NKVVRFDKLPGFGDS

SEQ ID NO: 297 - protein, 15 aa, synthetic construct
VRFDKLPGFGDSIEA

SEQ ID NO: 298 - protein, 15 aa, synthetic construct
DKLPGFGDSIEAQCG

SEQ ID NO: 299 - protein, 15 aa, synthetic construct
PGFGDSIEAQCGTSV

SEQ ID NO: 300 - protein, 15 aa, synthetic construct
GDSIEAQCGTSVNVH

SEQ ID NO: 301 - protein, 15 aa, synthetic construct
IEAQCGTSVNVHSSL

SEQ ID NO: 302 - protein, 15 aa, synthetic construct
QCGTSVNVHSSLRDI

SEQ ID NO: 303 - protein, 15 aa, synthetic construct
TSVNVHSSLRDILNQ

SEQ ID NO: 304 - protein, 15 aa, synthetic construct
NVHSSLRDILNQITK

SEQ ID NO: 305 - protein, 15 aa, synthetic construct
SSLRDILNQITKPND

SEQ ID NO: 306 - protein, 15 aa, synthetic construct
RDILNQITKPNDVYS

SEQ ID NO: 307 - protein, 15 aa, synthetic construct
LNQITKPNDVYSFSL

SEQ ID NO: 308 - protein, 15 aa, synthetic construct
ITKPNDVYSFSLASR

SEQ ID NO: 309 - protein, 15 aa, synthetic construct
PNDVYSFSLASRLYA

SEQ ID NO: 310 - protein, 15 aa, synthetic construct
VYSFSLASRLYAEER

SEQ ID NO: 311 - protein, 15 aa, synthetic construct
FSLASRLYAEERYPI

SEQ ID NO: 312 - protein, 15 aa, synthetic construct
ASRLYAEERYPILPE

SEQ ID NO: 313 - protein, 15 aa, synthetic construct
LYAEERYPILPEYLQ

SEQ ID NO: 314 - protein, 15 aa, synthetic construct
EERYPILPEYLQCVK

SEQ ID NO: 315 - protein, 15 aa, synthetic construct
YPILPEYLQCVKELY

SEQ ID NO: 316 - protein, 15 aa, synthetic construct
LPEYLQCVKELYRGG

SEQ ID NO: 317 - protein, 15 aa, synthetic construct
YLQCVKELYRGGLEP

SEQ ID NO: 318 - protein, 15 aa, synthetic construct
CVKELYRGGLEPINF

SEQ ID NO: 319 - protein, 15 aa, synthetic construct
ELYRGGLEPINFQTA

SEQ ID NO: 320 - protein, 15 aa, synthetic construct
RGGLEPINFQTAADQ

SEQ ID NO: 321 - protein, 15 aa, synthetic construct
LEPINFQTAADQARE

SEQ ID NO: 322 - protein, 15 aa, synthetic construct
INFQTAADQARELIN

SEQ ID NO: 323 - protein, 15 aa, synthetic construct
QTAADQARELINSWV

SEQ ID NO: 324 - protein, 15 aa, synthetic construct
ADQARELINSWVESQ

SEQ ID NO: 325 - protein, 15 aa, synthetic construct
ARELINSWVESQTNG

SEQ ID NO: 326 - protein, 15 aa, synthetic construct
LINSWVESQTNGIIR

SEQ ID NO: 327 - protein, 15 aa, synthetic construct
SWVESQTNGIIRNVL

SEQ ID NO: 328 - protein, 15 aa, synthetic construct
ESQTNGIIRNVLQPS

SEQ ID NO: 329 - protein, 15 aa, synthetic construct
TNGIIRNVLQPSSVD

SEQ ID NO: 330 - protein, 15 aa, synthetic construct
IIRNVLQPSSVDSQT

SEQ ID NO: 331 - protein, 15 aa, synthetic construct
NVLQPSSVDSQTAMV

SEQ ID NO: 332 - protein, 15 aa, synthetic construct
QPSSVDSQTAMVLVN

SEQ ID NO: 333 - protein, 15 aa, synthetic construct
SVDSQTAMVLVNAIV

SEQ ID NO: 334 - protein, 15 aa, synthetic construct
SQTAMVLVNAIVFKG

SEQ ID NO: 335 - protein, 15 aa, synthetic construct
AMVLVNAIVFKGLWE

SEQ ID NO: 336 - protein, 15 aa, synthetic construct
LVNAIVFKGLWEKAF

SEQ ID NO: 337 - protein, 15 aa, synthetic construct
AIVFKGLWEKAFKDE

SEQ ID NO: 338 - protein, 15 aa, synthetic construct
FKGLWEKAFKDEDTQ

SEQ ID NO: 339 - protein, 15 aa, synthetic construct
LWEKAFKDEDTQAMP

SEQ ID NO: 340 - protein, 15 aa, synthetic construct
KAFKDEDTQAMPFRV

SEQ ID NO: 341 - protein, 15 aa, synthetic construct
KDEDTQAMPFRVTEQ

SEQ ID NO: 342 - protein, 15 aa, synthetic construct
DTQAMPFRVTEQESK

SEQ ID NO: 343 - protein, 15 aa, synthetic construct
AMPFRVTEQESKPVQ

SEQ ID NO: 344 - protein, 15 aa, synthetic construct
FRVTEQESKPVQMMY

SEQ ID NO: 345 - protein, 15 aa, synthetic construct
TEQESKPVQMMYQIG

SEQ ID NO: 346 - protein, 15 aa, synthetic construct
ESKPVQMMYQIGLFR

SEQ ID NO: 347 - protein, 15 aa, synthetic construct
PVQMMYQIGLFRVAS

SEQ ID NO: 348 - protein, 15 aa, synthetic construct
MMYQIGLFRVASMAS

SEQ ID NO: 349 - protein, 15 aa, synthetic construct
QIGLFRVASMASEKM

SEQ ID NO: 350 - protein, 15 aa, synthetic construct
LFRVASMASEKMKIL

SEQ ID NO: 351 - protein, 15 aa, synthetic construct
VASMASEKMKILELP

SEQ ID NO: 352 - protein, 15 aa, synthetic construct
MASEKMKILELPFAS

SEQ ID NO: 353 - protein, 15 aa, synthetic construct
EKMKILELPFASGTM

SEQ ID NO: 354 - protein, 15 aa, synthetic construct
KILELPFASGTMSML

SEQ ID NO: 355 - protein, 15 aa, synthetic construct
ELPFASGTMSMLVLL

SEQ ID NO: 356 - protein, 15 aa, synthetic construct
FASGTMSMLVLLPDE

SEQ ID NO: 357 - protein, 15 aa, synthetic construct
GTMSMLVLLPDEVSG

SEQ ID NO: 358 - protein, 15 aa, synthetic construct
SMLVLLPDEVSGLEQ

SEQ ID NO: 359 - protein, 15 aa, synthetic construct
VLLPDEVSGLEQLES

SEQ ID NO: 360 - protein, 15 aa, synthetic construct
PDEVSGLEQLESIIN

SEQ ID NO: 361 - protein, 15 aa, synthetic construct
VSGLEQLESIINFEK

SEQ ID NO: 362 - protein, 15 aa, synthetic construct
LEQLESIINFEKLTE

SEQ ID NO: 363 - protein, 15 aa, synthetic construct
LESIINFEKLTEWTS

SEQ ID NO: 364 - protein, 15 aa, synthetic construct
IINFEKLTEWTSSNV

SEQ ID NO: 365 - protein, 15 aa, synthetic construct
FEKLTEWTSSNVMEE

SEQ ID NO: 366 - protein, 15 aa, synthetic construct
LTEWTSSNVMEERKI

SEQ ID NO: 367 - protein, 15 aa, synthetic construct
WTSSNVMEERKIKVY

SEQ ID NO: 368 - protein, 15 aa, synthetic construct
SNVMEERKIKVYLPR

SEQ ID NO: 369 - protein, 15 aa, synthetic construct
MEERKIKVYLPRMKM

SEQ ID NO: 370 - protein, 15 aa, synthetic construct
RKIKVYLPRMKMEEK

SEQ ID NO: 371 - protein, 15 aa, synthetic construct
KVYLPRMKMEEKYNL

SEQ ID NO: 372 - protein, 15 aa, synthetic construct
LPRMKMEEKYNLTSV

SEQ ID NO: 373 - protein, 15 aa, synthetic construct
MKMEEKYNLTSVLMA

SEQ ID NO: 374 - protein, 15 aa, synthetic construct
EEKYNLTSVLMAMGI

SEQ ID NO: 375 - protein, 15 aa, synthetic construct
YNLTSVLMAMGITDV

SEQ ID NO: 376 - protein, 15 aa, synthetic construct
TSVLMAMGITDVFSS

SEQ ID NO: 377 - protein, 15 aa, synthetic construct
LMAMGITDVFSSSAN

SEQ ID NO: 378 - protein, 15 aa, synthetic construct
MGITDVFSSSANLSG

SEQ ID NO: 379 - protein, 15 aa, synthetic construct
TDVFSSSANLSGISS

SEQ ID NO: 380 - protein, 15 aa, synthetic construct
FSSSANLSGISSAES

SEQ ID NO: 381 - protein, 15 aa, synthetic construct
SANLSGISSAESLKI

SEQ ID NO: 382 - protein, 15 aa, synthetic construct
LSGISSAESLKISQA

SEQ ID NO: 383 - protein, 15 aa, synthetic construct
ISSAESLKISQAVHA

SEQ ID NO: 384 - protein, 15 aa, synthetic construct
AESLKISQAVHAAHA

SEQ ID NO: 385 - protein, 15 aa, synthetic construct
LKISQAVHAAHAEIN

SEQ ID NO: 386 - protein, 15 aa, synthetic construct
SQAVHAAHAEINEAG

SEQ ID NO: 387 - protein, 15 aa, synthetic construct
VHAAHAEINEAGREV

SEQ ID NO: 388 - protein, 15 aa, synthetic construct
AHAEINEAGREVVGS

SEQ ID NO: 389 - protein, 15 aa, synthetic construct
EINEAGREVVGSAEA

SEQ ID NO: 390 - protein, 15 aa, synthetic construct
EAGREVVGSAEAGVD

SEQ ID NO: 391 - protein, 15 aa, synthetic construct
REVVGSAEAGVDAAS

SEQ ID NO: 392 - protein, 15 aa, synthetic construct
VGSAEAGVDAASVSE

SEQ ID NO: 393 - protein, 15 aa, synthetic construct
AEAGVDAASVSEEFR

SEQ ID NO: 394 - protein, 15 aa, synthetic construct
GVDAASVSEEFRADH

SEQ ID NO: 395 - protein, 15 aa, synthetic construct
AASVSEEFRADHPFL

SEQ ID NO: 396 - protein, 15 aa, synthetic construct
VSEEFRADHPFLFCI

SEQ ID NO: 397 - protein, 15 aa, synthetic construct
EFRADHPFLFCIKHI

SEQ ID NO: 398 - protein, 15 aa, synthetic construct
ADHPFLFCIKHIATN

SEQ ID NO: 399 - protein, 15 aa, synthetic construct
PFLFCIKHIATNAVL

SEQ ID NO: 400 - protein, 15 aa, synthetic construct
FCIKHIATNAVLFFG

SEQ ID NO: 401 - protein, 15 aa, synthetic construct
KHIATNAVLFFGRCV

SEQ ID NO: 402 - protein, 14 aa, synthetic construct
ATNAVLFFGRCVSP

SEQ ID NO: 403 - protein, 15 aa, synthetic construct
AEVDCSRFPNATDKE

SEQ ID NO: 404 - protein, 15 aa, synthetic construct
DCSRFPNATDKEGKD

SEQ ID NO: 405 - protein, 15 aa, synthetic construct
RFPNATDKEGKDVLV

SEQ ID NO: 406 - protein, 15 aa, synthetic construct
NATDKEGKDVLVCNK

SEQ ID NO: 407 - protein, 15 aa, synthetic construct
DKEGKDVLVCNKDLR

SEQ ID NO: 408 - protein, 15 aa, synthetic construct
GKDVLVCNKDLRPIC

SEQ ID NO: 409 - protein, 15 aa, synthetic construct
VLVCNKDLRPICGTD

SEQ ID NO: 410 - protein, 15 aa, synthetic construct
CNKDLRPICGTDGVT

SEQ ID NO: 411 - protein, 15 aa, synthetic construct
DLRPICGTDGVTYTN

SEQ ID NO: 412 - protein, 15 aa, synthetic construct
PICGTDGVTYTNDCL

SEQ ID NO: 413 - protein, 15 aa, synthetic construct
GTDGVTYTNDCLLCA

SEQ ID NO: 414 - protein, 15 aa, synthetic construct
GVTYTNDCLLCAYSI

SEQ ID NO: 415 - protein, 15 aa, synthetic construct
YTNDCLLCAYSIEFG

SEQ ID NO: 416 - protein, 15 aa, synthetic construct
DCLLCAYSIEFGTNI

SEQ ID NO: 417 - protein, 15 aa, synthetic construct
LCAYSIEFGTNISKE

SEQ ID NO: 418 - protein, 15 aa, synthetic construct
YSIEFGTNISKEHDG

SEQ ID NO: 419 - protein, 15 aa, synthetic construct
EFGTNISKEHDGECK

SEQ ID NO: 420 - protein, 15 aa, synthetic construct
TNISKEHDGECKETV

SEQ ID NO: 421 - protein, 15 aa, synthetic construct
SKEHDGECKETVPMN

SEQ ID NO: 422 - protein, 15 aa, synthetic construct
HDGECKETVPMNCSS

SEQ ID NO: 423 - protein, 15 aa, synthetic construct
ECKETVPMNCSSYAN

SEQ ID NO: 424 - protein, 15 aa, synthetic construct
ETVPMNCSSYANTTS

SEQ ID NO: 425 - protein, 15 aa, synthetic construct
PMNCSSYANTTSEDG

SEQ ID NO: 426 - protein, 15 aa, synthetic construct
CSSYANTTSEDGKVM

SEQ ID NO: 427 - protein, 15 aa, synthetic construct
YANTTSEDGKVMVLC

SEQ ID NO: 428 - protein, 15 aa, synthetic construct
TTSEDGKVMVLCNRA

SEQ ID NO: 429 - protein, 15 aa, synthetic construct
EDGKVMVLCNRAFNP

SEQ ID NO: 430 - protein, 15 aa, synthetic construct
KVMVLCNRAFNPVCG

SEQ ID NO: 431 - protein, 15 aa, synthetic construct
VLCNRAFNPVCGTDG

SEQ ID NO: 432 - protein, 15 aa, synthetic construct
NRAFNPVCGTDGVTY

SEQ ID NO: 433 - protein, 15 aa, synthetic construct
FNPVCGTDGVTYDNE

SEQ ID NO: 434 - protein, 15 aa, synthetic construct
VCGTDGVTYDNECLL

SEQ ID NO: 435 - protein, 15 aa, synthetic construct
TDGVTYDNECLLCAH

SEQ ID NO: 436 - protein, 15 aa, synthetic construct
VTYDNECLLCAHKVE

SEQ ID NO: 437 - protein, 15 aa, synthetic construct
DNECLLCAHKVEQGA

SEQ ID NO: 438 - protein, 15 aa, synthetic construct
CLLCAHKVEQGASVD

SEQ ID NO: 439 - protein, 15 aa, synthetic construct
CAHKVEQGASVDKRH

SEQ ID NO: 440 - protein, 15 aa, synthetic construct
KVEQGASVDKRHDGG

SEQ ID NO: 441 - protein, 15 aa, synthetic construct
QGASVDKRHDGGCRK

SEQ ID NO: 442 - protein, 15 aa, synthetic construct
SVDKRHDGGCRKELA

SEQ ID NO: 443 - protein, 15 aa, synthetic construct
KRHDGGCRKELAAVS

SEQ ID NO: 444 - protein, 15 aa, synthetic construct
DGGCRKELAAVSVDC

SEQ ID NO: 445 - protein, 15 aa, synthetic construct
CRKELAAVSVDCSEY

SEQ ID NO: 446 - protein, 15 aa, synthetic construct
ELAAVSVDCSEYPKP

SEQ ID NO: 447 - protein, 15 aa, synthetic construct
AVSVDCSEYPKPDCT

SEQ ID NO: 448 - protein, 15 aa, synthetic construct
VDCSEYPKPDCTAED

SEQ ID NO: 449 - protein, 15 aa, synthetic construct
SEYPKPDCTAEDRPL

SEQ ID NO: 450 - protein, 15 aa, synthetic construct
PKPDCTAEDRPLCGS

SEQ ID NO: 451 - protein, 15 aa, synthetic construct
DCTAEDRPLCGSDNK

SEQ ID NO: 452 - protein, 15 aa, synthetic construct
AEDRPLCGSDNKTYG

SEQ ID NO: 453 - protein, 15 aa, synthetic construct
RPLCGSDNKTYGNKC

SEQ ID NO: 454 - protein, 15 aa, synthetic construct
CGSDNKTYGNKCNFC

SEQ ID NO: 455 - protein, 15 aa, synthetic construct
DNKTYGNKCNFCNAV

SEQ ID NO: 456 - protein, 15 aa, synthetic construct
TYGNKCNFCNAVVES

SEQ ID NO: 457 - protein, 15 aa, synthetic construct
NKCNFCNAVVESNGT

SEQ ID NO: 458 - protein, 15 aa, synthetic construct
NFCNAVVESNGTLTL

SEQ ID NO: 459 - protein, 15 aa, synthetic construct
NAVVESNGTLTLSHF

SEQ ID NO: 460 - protein, 15 aa, synthetic construct
VESNGTLTLSHFGKC

SEQ ID NO: 461 - protein, 15 aa, synthetic construct
MADAAVIEKLEAGFK

SEQ ID NO: 462 - protein, 15 aa, synthetic construct
VIEKLEAGFKKLEAA

SEQ ID NO: 463 - protein, 15 aa, synthetic construct
EAGFKKLEAATDCKS

SEQ ID NO: 464 - protein, 15 aa, synthetic construct
KLEAATDCKSLLKKY

SEQ ID NO: 465 - protein, 15 aa, synthetic construct
TDCKSLLKKYLTKEV

SEQ ID NO: 466 - protein, 15 aa, synthetic construct
LLKKYLTKEVFDKLK

SEQ ID NO: 467 - protein, 15 aa, synthetic construct
LLKKYLTKEVFDKLK

SEQ ID NO: 468 - protein, 15 aa, synthetic construct
FDKLKDKKTSLGATL

SEQ ID NO: 469 - protein, 15 aa, synthetic construct
DKKTSLGATLLDVIQ

SEQ ID NO: 470 - protein, 15 aa, synthetic construct
LGATLLDVIQSGVEN

SEQ ID NO: 471 - protein, 15 aa, synthetic construct
LDVIQSGVENLDSGV

SEQ ID NO: 472 - protein, 15 aa, synthetic construct
SGVENLDSGVGIYAP

SEQ ID NO: 473 - protein, 15 aa, synthetic construct
LDSGVGIYAPDAEAY

SEQ ID NO: 474 - protein, 15 aa, synthetic construct
GIYAPDAEAYTLFAP

SEQ ID NO: 475 - protein, 15 aa, synthetic construct
DAEAYTLFAPLFDPI

SEQ ID NO: 476 - protein, 15 aa, synthetic construct
TLFAPLFDPIIEDYH

SEQ ID NO: 477 - protein, 15 aa, synthetic construct
LFDPIIEDYHVGFKQ

SEQ ID NO: 478 - protein, 15 aa, synthetic construct
IEDYHVGFKQTDKHP

SEQ ID NO: 479 - protein, 15 aa, synthetic construct
VGFKQTDKHPNKDFG

SEQ ID NO: 480 - protein, 15 aa, synthetic construct
TDKHPNKDFGDVNSF

SEQ ID NO: 481 - protein, 15 aa, synthetic construct
NKDFGDVNSFVNVDP

SEQ ID NO: 482 - protein, 15 aa, synthetic construct
DVNSFVNVDPEGKFV

SEQ ID NO: 483 - protein, 15 aa, synthetic construct
VNVDPEGKFVISTRV

SEQ ID NO: 484 - protein, 15 aa, synthetic construct
EGKFVISTRVRCGRS

SEQ ID NO: 485 - protein, 15 aa, synthetic construct
ISTRVRCGRSMQGYP

SEQ ID NO: 486 - protein, 15 aa, synthetic construct
RCGRSMQGYPFNPCL

SEQ ID NO: 487 - protein, 15 aa, synthetic construct
MQGYPFNPCLTESQY

SEQ ID NO: 488 - protein, 15 aa, synthetic construct
FNPCLTESQYKEMEA

SEQ ID NO: 489 - protein, 15 aa, synthetic construct
TESQYKEMEAKVSST

SEQ ID NO: 490 - protein, 15 aa, synthetic construct
KEMEAKVSSTLSSLE

SEQ ID NO: 491 - protein, 15 aa, synthetic construct
KVSSTLSSLEGELKG

SEQ ID NO: 492 - protein, 15 aa, synthetic construct
LSSLEGELKGTYYPL

SEQ ID NO: 493 - protein, 15 aa, synthetic construct
GELKGTYYPLTGMSK

SEQ ID NO: 494 - protein, 15 aa, synthetic construct
TYYPLTGMSKEVQQK

SEQ ID NO: 495 - protein, 15 aa, synthetic construct
TGMSKEVQQKLIDDH

SEQ ID NO: 496 - protein, 15 aa, synthetic construct
EVQQKLIDDHFLFKE

SEQ ID NO: 497 - protein, 15 aa, synthetic construct
LIDDHFLFKEGDRFL

SEQ ID NO: 498 - protein, 15 aa, synthetic construct
FLFKEGDRFLQAANA

SEQ ID NO: 499 - protein, 15 aa, synthetic construct
GDRFLQAANACRYWP

SEQ ID NO: 500 - protein, 15 aa, synthetic construct
QAANACRYWPAGRGI

SEQ ID NO: 501 - protein, 15 aa, synthetic construct
CRYWPAGRGIYHNDN

SEQ ID NO: 502 - protein, 15 aa, synthetic construct
AGRGIYHNDNKTFLV

SEQ ID NO: 503 - protein, 15 aa, synthetic construct
YHNDNKTFLVWVNEE

SEQ ID NO: 504 - protein, 15 aa, synthetic construct
KTFLVWVNEEDHLRI

SEQ ID NO: 505 - protein, 15 aa, synthetic construct
WVNEEDHLRIISMQM

SEQ ID NO: 506 - protein, 15 aa, synthetic construct
DHLRIISMQMGGDLG

SEQ ID NO: 507 - protein, 15 aa, synthetic construct
ISMQMGGDLGQVFRR

SEQ ID NO: 508 - protein, 15 aa, synthetic construct
GGDLGQVFRRLTSAV

SEQ ID NO: 509 - protein, 15 aa, synthetic construct
QVFRRLTSAVNEIEK

SEQ ID NO: 510 - protein, 15 aa, synthetic construct
LTSAVNEIEKRIPFS

SEQ ID NO: 511 - protein, 15 aa, synthetic construct
NEIEKRIPFSHHDRL

SEQ ID NO: 512 - protein, 15 aa, synthetic construct
RIPFSHHDRLGFLTF

SEQ ID NO: 513 - protein, 15 aa, synthetic construct
HHDRLGFLTFCPTNL

SEQ ID NO: 514 - protein, 15 aa, synthetic construct
GFLTFCPTNLGTTVR

SEQ ID NO: 515 - protein, 15 aa, synthetic construct
CPTNLGTTVRASVHI

SEQ ID NO: 516 - protein, 15 aa, synthetic construct
GTTVRASVHIKLPKL

SEQ ID NO: 517 - protein, 15 aa, synthetic construct
ASVHIKLPKLAANRE

SEQ ID NO: 518 - protein, 15 aa, synthetic construct
KLPKLAANREKLEEV

SEQ ID NO: 519 - protein, 15 aa, synthetic construct
AANREKLEEVAGKYN

SEQ ID NO: 520 - protein, 15 aa, synthetic construct
KLEEVAGKYNLQVRG

SEQ ID NO: 521 - protein, 15 aa, synthetic construct
AGKYNLQVRGTRGEH

SEQ ID NO: 522 - protein, 15 aa, synthetic construct
LQVRGTRGEHTEAEG

SEQ ID NO: 523 - protein, 15 aa, synthetic construct
TRGEHTEAEGGIYDI

SEQ ID NO: 524 - protein, 15 aa, synthetic construct
TEAEGGIYDISNKRR

SEQ ID NO: 525 - protein, 15 aa, synthetic construct
GIYDISNKRRMGLTE

SEQ ID NO: 526 - protein, 15 aa, synthetic construct
SNKRRMGLTEFQAVK

SEQ ID NO: 527 - protein, 15 aa, synthetic construct
MGLTEFQAVKEMQDG

SEQ ID NO: 528 - protein, 15 aa, synthetic construct
FQAVKEMQDGILELI

SEQ ID NO: 529 - protein, 15 aa, synthetic construct
EMQDGILELIKIEKE

SEQ ID NO: 530 - protein, 15 aa, synthetic construct
MSRKSGSRSSSKRSK

SEQ ID NO: 531 - protein, 15 aa, synthetic construct
GSRSSSKRSKKSGGG

SEQ ID NO: 532 - protein, 15 aa, synthetic construct
SKRSKKSGGGSNVFD

SEQ ID NO: 533 - protein, 15 aa, synthetic construct
KSGGGSNVFDMFTQR

SEQ ID NO: 534 - protein, 15 aa, synthetic construct
SNVFDMFTQRQVAEF

SEQ ID NO: 535 - protein, 15 aa, synthetic construct
MFTQRQVAEFKEGFQ

SEQ ID NO: 536 - protein, 15 aa, synthetic construct
QVAEFKEGFQLMDRD

SEQ ID NO: 537 - protein, 15 aa, synthetic construct
KEGFQLMDRDKDGVI

SEQ ID NO: 538 - protein, 15 aa, synthetic construct
LMDRDKDGVIGKTDL

SEQ ID NO: 539 - protein, 15 aa, synthetic construct
KDGVIGKTDLRGTFD

SEQ ID NO: 540 - protein, 15 aa, synthetic construct
GKTDLRGTFDEIGRI

SEQ ID NO: 541 - protein, 15 aa, synthetic construct
RGTFDEIGRIATDQE

SEQ ID NO: 542 - protein, 15 aa, synthetic construct
EIGRIATDQELDEML

SEQ ID NO: 543 - protein, 15 aa, synthetic construct
ATDQELDEMLADAPA

SEQ ID NO: 544 - protein, 15 aa, synthetic construct
LDEMLADAPAPINFT

SEQ ID NO: 545 - protein, 15 aa, synthetic construct
ADAPAPINFTMLLNM

SEQ ID NO: 546 - protein, 15 aa, synthetic construct
PINFTMLLNMFAERQ

SEQ ID NO: 547 - protein, 15 aa, synthetic construct
MLLNMFAERQTGESD

SEQ ID NO: 548 - protein, 15 aa, synthetic construct
FAERQTGESDDDDVV

SEQ ID NO: 549 - protein, 15 aa, synthetic construct
TGESDDDDVVAKAFL

SEQ ID NO: 550 - protein, 15 aa, synthetic construct
DDDVVAKAFLAFADE

SEQ ID NO: 551 - protein, 15 aa, synthetic construct
AKAFLAFADEEGNID

SEQ ID NO: 552 - protein, 15 aa, synthetic construct
AFADEEGNIDCDTFR

SEQ ID NO: 553 - protein, 15 aa, synthetic construct
EGNIDCDTFRHALMT

SEQ ID NO: 554 - protein, 15 aa, synthetic construct
CDTFRHALMTWGDKF

SEQ ID NO: 555 - protein, 15 aa, synthetic construct
HALMTWGDKFSSQEA

SEQ ID NO: 556 - protein, 15 aa, synthetic construct
WGDKFSSQEADDALD

SEQ ID NO: 557 - protein, 15 aa, synthetic construct
SSQEADDALDQMDID

SEQ ID NO: 558 - protein, 15 aa, synthetic construct
DDALDQMDIDDGGKI

SEQ ID NO: 559 - protein, 15 aa, synthetic construct
QMDIDDGGKIDVQGV

SEQ ID NO: 560 - protein, 15 aa, synthetic construct
DGGKIDVQGVIQMLT

SEQ ID NO: 561 - protein, 15 aa, synthetic construct
DVQGVIQMLTAGGGD

SEQ ID NO: 562 - protein, 15 aa, synthetic construct
IQMLTAGGGDDAAAE

SEQ ID NO: 563 - protein, 12 aa, synthetic construct
AGGGDDAAAEEA

SEQ ID NO: 564 - protein, 15 aa, synthetic construct
MAYSWDNRVKYVVRY

SEQ ID NO: 565 - protein, 15 aa, synthetic construct
DNRVKYVVRYMYDID

SEQ ID NO: 566 - protein, 15 aa, synthetic construct
YVVRYMYDIDNNGFL

SEQ ID NO: 567 - protein, 15 aa, synthetic construct
MYDIDNNGFLDKNDF

SEQ ID NO: 568 - protein, 15 aa, synthetic construct
NNGFLDKNDFECLAV

SEQ ID NO: 569 - protein, 15 aa, synthetic construct
DKNDFECLAVRNTLI

SEQ ID NO: 570 - protein, 15 aa, synthetic construct
ECLAVRNTLIEGRGE

SEQ ID NO: 571 - protein, 15 aa, synthetic construct
RNTLIEGRGEFSADA

SEQ ID NO: 572 - protein, 15 aa, synthetic construct
EGRGEFSADAYANNQ

SEQ ID NO: 573 - protein, 15 aa, synthetic construct
FSADAYANNQKIMRN

SEQ ID NO: 574 - protein, 15 aa, synthetic construct
YANNQKIMRNLWNEI

SEQ ID NO: 575 - protein, 15 aa, synthetic construct
KIMRNLWNEIAELAD

SEQ ID NO: 576 - protein, 15 aa, synthetic construct
LWNEIAELADFNKDG

SEQ ID NO: 577 - protein, 15 aa, synthetic construct
AELADFNKDGEVTVD

SEQ ID NO: 578 - protein, 15 aa, synthetic construct
FNKDGEVTVDEFKQA

SEQ ID NO: 579 - protein, 15 aa, synthetic construct
EVTVDEFKQAVQKHC

SEQ ID NO: 580 - protein, 15 aa, synthetic construct
EFKQAVQKHCQGKKY

SEQ ID NO: 581 - protein, 15 aa, synthetic construct
VQKHCQGKKYGDFPG

SEQ ID NO: 582 - protein, 15 aa, synthetic construct
QGKKYGDFPGAFKVF

SEQ ID NO: 583 - protein, 15 aa, synthetic construct
GDFPGAFKVFIANQF

SEQ ID NO: 584 - protein, 15 aa, synthetic construct
AFKVFIANQFKAIDV

SEQ ID NO: 585 - protein, 15 aa, synthetic construct
IANQFKAIDVNGDGK

SEQ ID NO: 586 - protein, 15 aa, synthetic construct
KAIDVNGDGKVGLDE

SEQ ID NO: 587 - protein, 15 aa, synthetic construct
NGDGKVGLDEYRLDC

SEQ ID NO: 588 - protein, 15 aa, synthetic construct
VGLDEYRLDCITRSA

SEQ ID NO: 589 - protein, 15 aa, synthetic construct
YRLDCITRSAFAEVK

SEQ ID NO: 590 - protein, 15 aa, synthetic construct
ITRSAFAEVKEIDDA

SEQ ID NO: 591 - protein, 15 aa, synthetic construct
FAEVKEIDDAYNKLT

SEQ ID NO: 592 - protein, 15 aa, synthetic construct
EIDDAYNKLTTEDDR

SEQ ID NO: 593 - protein, 15 aa, synthetic construct
YNKLTTEDDRKAGGL

SEQ ID NO: 594 - protein, 15 aa, synthetic construct
TEDDRKAGGLTLERY

SEQ ID NO: 595 - protein, 15 aa, synthetic construct
KAGGLTLERYQDLYA

SEQ ID NO: 596 - protein, 15 aa, synthetic construct
TLERYQDLYAQFISN

SEQ ID NO: 597 - protein, 15 aa, synthetic construct
QDLYAQFISNPDESC

SEQ ID NO: 598 - protein, 15 aa, synthetic construct
QFISNPDESCSACYL

SEQ ID NO: 599 - protein, 15 aa, synthetic construct
PDESCSACYLFGPLK

SEQ ID NO: 600 - protein, 13 aa, synthetic construct
SACYLFGPLKVVQ

SEQ ID NO: 601 - protein, 15 aa, synthetic construct
MDAIKKKMQAMKLEK

SEQ ID NO: 602 - protein, 15 aa, synthetic construct
KKMQAMKLEKDNAMD

SEQ ID NO: 603 - protein, 15 aa, synthetic construct
MKLEKDNAMDRADTL

SEQ ID NO: 604 - protein, 15 aa, synthetic construct
DNAMDRADTLEQQNK

SEQ ID NO: 605 - protein, 15 aa, synthetic construct
RADTLEQQNKEANNR

SEQ ID NO: 606 - protein, 15 aa, synthetic construct
EQQNKEANNRAEKSE

SEQ ID NO: 607 - protein, 15 aa, synthetic construct
EANNRAEKSEEEVHN

SEQ ID NO: 608 - protein, 15 aa, synthetic construct
AEKSEEEVHNLQKRM

SEQ ID NO: 609 - protein, 15 aa, synthetic construct
EEVHNLQKRMQQLEN

SEQ ID NO: 610 - protein, 15 aa, synthetic construct
LQKRMQQLENDLDQV

SEQ ID NO: 611 - protein, 15 aa, synthetic construct
QQLENDLDQVQESLL

SEQ ID NO: 612 - protein, 15 aa, synthetic construct
DLDQVQESLLKANIQ

SEQ ID NO: 613 - protein, 15 aa, synthetic construct
QESLLKANIQLVEKD

SEQ ID NO: 614 - protein, 15 aa, synthetic construct
KANIQLVEKDKALSN

SEQ ID NO: 615 - protein, 15 aa, synthetic construct
LVEKDKALSNAEGEV

SEQ ID NO: 616 - protein, 15 aa, synthetic construct
KALSNAEGEVAALNR

SEQ ID NO: 617 - protein, 15 aa, synthetic construct
AEGEVAALNRRIQLL

SEQ ID NO: 618 - protein, 15 aa, synthetic construct
AALNRRIQLLEEDLE

SEQ ID NO: 619 - protein, 15 aa, synthetic construct
RIQLLEEDLERSEER

SEQ ID NO: 620 - protein, 15 aa, synthetic construct
EEDLERSEERLNTAT

SEQ ID NO: 621 - protein, 15 aa, synthetic construct
RSEERLNTATTKLAE

SEQ ID NO: 622 - protein, 15 aa, synthetic construct
LNTATTKLAEASQAA

SEQ ID NO: 623 - protein, 15 aa, synthetic construct
TKLAEASQAADESER

SEQ ID NO: 624 - protein, 15 aa, synthetic construct
ASQAADESERMRKVL

SEQ ID NO: 625 - protein, 15 aa, synthetic construct
DESERMRKVLENRSL

SEQ ID NO: 626 - protein, 15 aa, synthetic construct
MRKVLENRSLSDEER

SEQ ID NO: 627 - protein, 15 aa, synthetic construct
ENRSLSDEERMDALE

SEQ ID NO: 628 - protein, 15 aa, synthetic construct
SDEERMDALENQLKE

SEQ ID NO: 629 - protein, 15 aa, synthetic construct
MDALENQLKEARFLA

SEQ ID NO: 630 - protein, 15 aa, synthetic construct
NQLKEARFLAEEADR

SEQ ID NO: 631 - protein, 15 aa, synthetic construct
ARFLAEEADRKYDEV

SEQ ID NO: 632 - protein, 15 aa, synthetic construct
EEADRKYDEVARKLA

SEQ ID NO: 633 - protein, 15 aa, synthetic construct
KYDEVARKLAMVEAD

SEQ ID NO: 634 - protein, 15 aa, synthetic construct
ARKLAMVEADLERAE

SEQ ID NO: 635 - protein, 15 aa, synthetic construct
MVEADLERAEERAET

SEQ ID NO: 636 - protein, 15 aa, synthetic construct
LERAEERAETGESKI

SEQ ID NO: 637 - protein, 15 aa, synthetic construct
ERAETGESKIVELEE

SEQ ID NO: 638 - protein, 15 aa, synthetic construct
GESKIVELEEELRVV

SEQ ID NO: 639 - protein, 15 aa, synthetic construct
VELEEELRVVGNNLK

SEQ ID NO: 640 - protein, 15 aa, synthetic construct
ELRVVGNNLKSLEVS

SEQ ID NO: 641 - protein, 15 aa, synthetic construct
GNNLKSLEVSEEKAN

SEQ ID NO: 642 - protein, 15 aa, synthetic construct
SLEVSEEKANQREEA

SEQ ID NO: 643 - protein, 15 aa, synthetic construct
EEKANQREEAYKEQI

SEQ ID NO: 644 - protein, 15 aa, synthetic construct
QREEAYKEQIKTLTN

SEQ ID NO: 645 - protein, 15 aa, synthetic construct
YKEQIKTLTNKLKAA

SEQ ID NO: 646 - protein, 15 aa, synthetic construct
KTLTNKLKAAEARAE

SEQ ID NO: 647 - protein, 15 aa, synthetic construct
KLKAAEARAEFAERS

SEQ ID NO: 648 - protein, 15 aa, synthetic construct
EARAEFAERSVQKLQ

SEQ ID NO: 649 - protein, 15 aa, synthetic construct
FAERSVQKLQKEVDR

SEQ ID NO: 650 - protein, 15 aa, synthetic construct
VQKLQKEVDRLEDEL

SEQ ID NO: 651 - protein, 15 aa, synthetic construct
KEVDRLEDELVNEKE

SEQ ID NO: 652 - protein, 15 aa, synthetic construct
LEDELVNEKEKYKSI

SEQ ID NO: 653 - protein, 15 aa, synthetic construct
VNEKEKYKSITDELD

SEQ ID NO: 654 - protein, 15 aa, synthetic construct
KYKSITDELDQTFSE

SEQ ID NO: 655 - protein, 14 aa, synthetic construct
TDELDQTFSELSGY

SEQ ID NO: 656 - protein, 15 aa, synthetic construct
MDSLDEEQIETLRKA

SEQ ID NO: 657 - protein, 15 aa, synthetic construct
EEQIETLRKAFDSFD

SEQ ID NO: 658 - protein, 15 aa, synthetic construct
TLRKAFDSFDTEKTG

SEQ ID NO: 659 - protein, 15 aa, synthetic construct
FDSFDTEKTGSITAE

SEQ ID NO: 660 - protein, 15 aa, synthetic construct
TEKTGSITAETIATI

SEQ ID NO: 661 - protein, 15 aa, synthetic construct
SITAETIATIMRMMG

SEQ ID NO: 662 - protein, 15 aa, synthetic construct
TIATIMRMMGVKISE

SEQ ID NO: 663 - protein, 15 aa, synthetic construct
MRMMGVKISEKNLQE

SEQ ID NO: 664 - protein, 15 aa, synthetic construct
VKISEKNLQEAIAET

SEQ ID NO: 665 - protein, 15 aa, synthetic construct
KNLQEAIAETDEDGS

SEQ ID NO: 666 - protein, 15 aa, synthetic construct
AIAETDEDGSGLLEF

SEQ ID NO: 667 - protein, 15 aa, synthetic construct
DEDGSGLLEFEEFVE

SEQ ID NO: 668 - protein, 15 aa, synthetic construct
GLLEFEEFVELSAKF

SEQ ID NO: 669 - protein, 15 aa, synthetic construct
EEFVELSAKFLIEED

SEQ ID NO: 670 - protein, 15 aa, synthetic construct
LSAKFLIEEDEEALK

SEQ ID NO: 671 - protein, 15 aa, synthetic construct
LIEEDEEALKAELRE

SEQ ID NO: 672 - protein, 15 aa, synthetic construct
EEALKAELREAFRIY

SEQ ID NO: 673 - protein, 15 aa, synthetic construct
AELREAFRIYDKEGN

SEQ ID NO: 674 - protein, 15 aa, synthetic construct
AFRIYDKEGNGFITT

SEQ ID NO: 675 - protein, 15 aa, synthetic construct
DKEGNGFITTDVLKE

SEQ ID NO: 676 - protein, 15 aa, synthetic construct
GFITTDVLKEILAEL

SEQ ID NO: 677 - protein, 15 aa, synthetic construct
DVLKEILAELDPRLT

SEQ ID NO: 678 - protein, 15 aa, synthetic construct
ILAELDPRLTPADLE

SEQ ID NO: 679 - protein, 15 aa, synthetic construct
DPRLTPADLENIIEE

SEQ ID NO: 680 - protein, 15 aa, synthetic construct
PADLENIIEEVDEDG

SEQ ID NO: 681 - protein, 15 aa, synthetic construct
NIIEEVDEDGSGTLD

SEQ ID NO: 682 - protein, 15 aa, synthetic construct
VDEDGSGTLDFDEFM

SEQ ID NO: 683 - protein, 15 aa, synthetic construct
SGTLDFDEFMEMMNG